(19) United States
(12) Patent Application Publication
(10) Pub. No.: US 2022/0113307 A1
(43) Pub. Date: Apr. 14, 2022

(54) RAPID DIAGNOSTIC TESTS UTILIZING TRAP REGIONS

(71) Applicant: Detect, Inc., Guilford, CT (US)

(72) Inventors: Amy Cornell, Killingworth, CT (US); John H. Leamon, Stonington, CT (US); Jonathan M. Rothberg, Miami Beach, FL (US); Henry Kemble, Paris (FR); James A. Ball, Ledyard, CT (US)

(73) Assignee: Detect, Inc., Guilford, CT (US)

(21) Appl. No.: 17/497,210

(22) Filed: Oct. 8, 2021

Related U.S. Application Data
(60) Provisional application No. 63/229,877, filed on Aug. 5, 2021.
(60) Provisional application No. 63/161,861, filed on Mar. 16, 2021.
(60) Provisional application No. 63/148,250, filed on Feb. 11, 2021.
(60) Provisional application No. 63/090,111, filed on Oct. 9, 2020.

(51) Int. Cl. G01N 33/543; G01N 33/53

(52) U.S. Cl. CPC G01N 33/54388; G01N 33/5308

(57) ABSTRACT

Aspects of the disclosure relate to compositions and methods for amplifying and/or detecting one or more target nucleic acid sequences (e.g., a nucleic acid sequence of one or more pathogens), and use of a trap region or reagent to reduce the level of one or more target nucleic acid analytes in an amplified sample prior to or in conjunction with detection. In some embodiments, the pathogens are viral, bacterial, fungal, parasitic, or protozoan pathogens, such as SARS-CoV-2 or an influenza virus. In some embodiments, the methods comprise isothermal amplification of a target nucleic acid and subsequent reduction in the level of one or more target nucleic acid analytes and detection of the amplification products.

### RELATED APPLICATION

This application claims the benefit under 35 U.S.C. 119(e) of U.S. provisional application No. 63/090,111 filed Oct. 9, 2020, U.S. provisional application No. 63/148,250 filed Feb. 11, 2021, U.S. provisional application No. 63/161,861 filed Mar. 16, 2021, and U.S. provisional application No. 63/229,877 filed Aug. 5, 2021, each of which is incorporated by reference herein in its entirety.

### FIELD

The disclosure generally relates to diagnostic devices, systems, and methods for detecting the presence of a target nucleic acid sequence.

### BACKGROUND

The ability to rapidly diagnose diseases—particularly highly infectious diseases—is critical to protecting human health. As one example, the high level of contagiousness, the high mortality rate, and the lack of a treatment for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease and treatment of infected individuals.

### SUMMARY

Aspects of the disclosure relate to compositions and methods for amplifying and/or detecting target analytes (e.g., nucleic acids) in a sample. The disclosure is based, in part, on immunoassay devices (e.g., lateral flow immunoassays, e.g., comprising lateral flow assay strips) that produce high contrast, visible bands when amplification products are applied thereto. In some embodiments, compositions and methods described herein allow for direct application of the amplicons to immunoassay devices without any intervening fluid transfer or dilution steps.

Accordingly, in some aspects, the disclosure provides a lateral flow assay strip usable to identify at least one target nucleic acid. In some embodiments, the lateral flow assay strip comprises an absorbent substrate having a first end and a second end, wherein the substrate is configured to move a fluid sample from the first end to the second end by capillary action. In some embodiments, the lateral assay strip comprises a plurality of sub-regions.

In some embodiments, the plurality of sub-regions comprises a first sub-region wherein the fluid sample can be introduced to the lateral flow assay strip. In some embodiments, the plurality of sub-regions comprises a second sub-region comprising a particle conjugate pad. In some embodiments, the plurality of sub-regions comprises a third sub-region comprising one or more test lines. In some embodiments, the one or more test lines are configured to indicate the presence of at least one target nucleic acid by interaction with the fluid sample. In some embodiments, the plurality of sub-regions comprises a fourth sub-region comprising a trap region. In some embodiments, the lateral flow assay strip does not comprise the first sub-region, and wherein the fluid sample can be introduced to the lateral flow assay strip in the second sub-region. In some embodiments, the sub-regions are arranged from the first end to the second end on the lateral flow assay strip in the order of: optionally the first sub-region, the fourth sub-region, optionally the second sub-region, and the third sub-region. In some embodiments, the sub-regions are arranged from the first end to the second end on the lateral flow assay strip in the order of: optionally the first sub-region, optionally the second sub-region, the fourth sub-region, and the third sub-region.

In some embodiments, the plurality of sub-regions comprises a first sub-region wherein the fluid sample can be introduced to the lateral flow assay strip. In some embodiments, the first sub-region comprises a trap region comprising a plurality of positively charged moieties capable of binding nucleic acid. In some embodiments, the plurality of sub-regions comprises a second sub-region comprising a particle conjugate pad. In some embodiments, the plurality of sub-regions comprises a third sub-region comprising one or more test lines. In some embodiments, the one or more test lines are configured to indicate the presence of at least one target nucleic acid by interaction with the fluid sample. In some embodiments, the sub-regions are arranged from the first end to the second end on the lateral flow assay strip in the order of: the first sub-region, optionally the second sub-region, and the third sub-region.

In some embodiments, the fluid sample comprises an amplification product mixture. In some embodiments, the amplification product mixture comprises labeled target amplification products, labeled control amplification products, and/or labeled unextended primers.

In some embodiments, the trap region comprises one or more trap lines or a trap pad. In some embodiments, the trap region comprises one or more trap reagents (e.g., one, two, three, four, five, six, seven, eight, nine, or ten different trap reagents).

In some embodiments, a trap region comprises a plurality of positively charged moieties. In some embodiments, the plurality of positively charged moieties comprise amine groups, e.g., primary amine groups, secondary amine groups, tertiary amine groups, quaternary amine groups, or any combination thereof. In some embodiments, the amine groups comprise an alkylamine, arylamine, and/or alkylarylamine group. Non-limiting examples of suitable amine groups include trimethylamino, dimethylamino, and diethylaminoethyl (DEAE) groups. In some embodiments, the positively charged moiety comprising an amine group comprises lysine. In some embodiments, the amine groups comprise a heterocyclic group comprising at least one nitrogen atom. Non-limiting examples of suitable heterocyclic groups include pyrrolidinyl, piperidinyl, and morpholino groups. In some embodiments, the heterocyclic group comprises an aromatic group (e.g., a heteroaromatic amine group). Non-limiting examples of suitable aromatic groups include pyrrolyl, imidazolyl, pyrazolyl, triazolyl, pyridinyl, and pyrimidinyl groups. In some embodiments, the plurality of positively charged moieties comprises an imine. Each amine or imine group described herein may be substituted or unsubstituted.

In some embodiments, the absorbent substrate and/or first sub-region comprises a polymer. In some embodiments, the polymer comprises an oligosaccharide or polysaccharide, e.g., cellulose, nitrocellulose, dextrin, chitin, chitosan, or agarose. In some embodiments, the oligosaccharide or polysaccharide comprises a plurality of sugar monomers (e.g., one, two, three, or all of a tetrose, a pentose, a hexose, or a heptose), wherein a plurality of the sugar monomers are chemically modified to comprise the plurality of positively charged moieties. In some embodiments, the plurality of sugar monomers comprise 1, 2, 3, 4, 5, 6, 7, or all of glucose, galactose, arabinose, mannose, fructose, xylose, fucose, and rhamnose. In some embodiments, the positively charged moieties are connected to the sugar monomers via one or more hydroxyl groups of the sugar monomers. In some embodiments, the positively charged moieties were attached by reacting the oligosaccharide or polysaccharide with a composition comprising an epoxide and an amine. In some embodiments, the reaction occurred under alkaline conditions. In some embodiments, the reaction occurred under acidic conditions. In some embodiments, the composition comprises glycidyltrimethylammonium chloride (GTMAC) and/or 2-chloro-N,N-diethylethanamine (also known as 2-chlorotriethylamine). In some embodiments, the polymer comprises an acrylic polymer and/or a styrenic polymer (e.g., polystyrene). In some embodiments, the acrylic polymer is a methyl acrylic polymer (e.g., poly(methyl acrylate), poly(methyl methacrylate)).

In some embodiments, the first sub-region (e.g., the absorbent substrate of the first sub-region) is thicker than at least one adjacent sub-region (e.g., the second, third, or fourth sub-region). In some embodiments, the first sub-region is at least 10, 20, 30, 40, 50, 60, 70, 80, 90, 100, 120, 140, 160, 180, 200, 220, 240, 260, 280, 300, 320, 340, 360, 380, 400, 420, 440, 460, 480, or 500% thicker than at least one adjacent sub-region, or is 10-500, 10-450, 10-400, 10-350, 10-300, 10-250, 10-200, 10-150, 10-100, 10-50, 50-500, 50-450, 50-400, 50-350, 50-300, 50-250, 50-200, 50-150, 50-100, 100-500, 100-450, 100-400, 100-350, 100-300, 100-250, 100-200, 100-150, 150-500, 150-450, 150-400, 150-350, 150-300, 150-250, 150-200, 200-500, 200-450, 200-400, 200-350, 200-300, 200-250, 250-500, 250-450, 250-400, 250-350, 250-300, 300-500, 300-450, 300-400, 300-350, 350-500, 350-450, 350-400, 400-500, 400-450, or 450-500% thicker than at least one adjacent sub-region. As used herein, “adjacent sub-region” refers to a sub-region that is immediately or directly adjacent to the sub-region in question (i.e., without another intervening sub-region between).

In some embodiments, the level of nucleic acid in a fluid sample after it has flowed through the trap region is decreased by at least 1, 5, 10, 20, 30, 40, 50, 60, 70, 80, or 90% relative to the level of nucleic acid present in the fluid sample prior to flowing through the trap region. In some embodiments, the level of nucleic acid in a fluid sample after it has flowed through the trap region is decreased by 1-90, 1-80, 1-70, 1-60, 1-50, 1-40, 1-30, 1-20, 1-10, 1-5, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90% relative to the level of nucleic acid present in the fluid sample prior to flowing through the trap region.

In some embodiments, a lateral flow assay strip is comprised within a diagnostic device.

In another aspect, the disclosure is directed, in part, to a method for detecting a target nucleic acid in a fluid sample, comprising contacting a lateral flow assay strip described herein with a fluid sample, e.g., comprising an amplification product mixture comprising labeled target amplification products, under conditions under which a target nucleic acid or labeled target amplification product binds to the first test line to produce a first detectable signal. In some embodiments, contacting occurs under conditions under which a labeled target amplification product which may be present in the amplification product mixture can bind to the one or more test lines to produce a first detectable signal. In some embodiments, the method further comprises identifying the presence of the target nucleic acid in the fluid sample based upon detecting the presence of the first detectable signal, or identifying the absence of the target nucleic acid in the fluid sample based on detecting the absence of the first detectable signal.

In some embodiments, the fluid sample further comprises labeled control amplification products. In some embodiments, the labeled control amplification products bind to the second test line to produce a second detectable signal. In some embodiments, identifying the presence or absence of the target nucleic acid in the fluid sample is further based upon detecting the presence of the second detectable signal.

In some embodiments, a fluid sample comprises an amplification product mixture comprising labeled target amplification products. In some embodiments, a method described herein further comprises performing an isothermal amplification reaction on a biological sample (e.g., a biological sample comprising a target gene), thereby producing an amplification product mixture comprising labeled target amplification products. In some embodiments, the biological sample further comprises a control gene, e.g., from which is produced a labeled control amplification product. In some embodiments, the amplification reaction is performed in a buffer solution comprising one or more biotinylated oligonucleotides, a target nucleic acid-specific primer comprising a first hapten, and a control nucleic acid-specific primer comprising a second hapten different from the first hapten. In some embodiments, the amplification product mixture further comprises labeled control amplification products. In some embodiments, contacting a lateral flow assay strip with a fluid sample occurs under conditions such that the labeled control amplification products bind to the second test line to produce a second detectable signal.

In some embodiments, a method described herein does not comprise diluting a fluid sample (e.g., a fluid sample comprising an amplification product mixture). In some embodiments, a method described herein does not comprise diluting an amplification product mixture. In some embodiments, the amplification product mixture or aliquot thereof used in a method described herein or applicable to a device described herein is undiluted. Without wishing to be bound by theory, the disclosure is directed, in part, to the discovery that dilution steps may be undesirable in point-of-care or at home testing settings and to methods and devices that enable rapid, accurate detection of target nucleic acids without dilution of samples.

### BRIEF DESCRIPTION OF THE DRAWINGS

FIGS. 1A-1D shows images of exemplary lateral flow assay strips run with samples comprising LAMP amplification reaction products spiked with anti-FITC monoclonal antibody (0.5 mg/mL mAb), with or without sample dilution. FIG. 1A shows undiluted LAMP samples, FIG. 1B. shows undiluted LAMP samples spiked with 2.5 μg mAb, FIG. 1C shows LAMP samples diluted 14×, and FIG. 1D shows LAMP samples diluted 14× and spiked with 2.5 μg mAb.

FIGS. 2A-2D shows images of exemplary lateral flow assay strips run with samples comprising LAMP amplification reaction products spiked with OD 10 of 40 nm gold nanoparticles (referred to as “OD 10 Gold”). FIG. 2A shows LAMP samples diluted 14×, FIG. 2B shows LAMP samples diluted 14× spiked with OD 10 Gold, FIG. 2C shows undiluted LAMP samples, and FIG. 2D. shows undiluted LAMP samples spiked with OD 10 Gold.

FIGS. 3A-3D show schematics of exemplary lateral flow assay strip layouts and an image of an exemplary lateral flow assay strip after running. FIG. 3A shows a schematic of a lateral flow assay strip configuration without a trap region. FIG. 3B shows a schematic of a lateral flow assay strip design utilizing a trap region. FIG. 3C shows a schematic of a lateral flow assay strip design utilizing a trap region upstream from the conjugate pad. FIG. 3D shows an exemplary LFA layout, left to right: LAMP reaction containing no template nucleic acid (No Template Control (NTC), LAMP reaction failing to amplify nucleic acid from a healthy RNAseP negative sample (−), and LAMP reaction amplifying nucleic acid from a COVID positive sample (+).

FIGS. 4A-4B show images of exemplary lateral flow assay strips run with samples of LAMP reaction amplifying nucleic acid from a COVID positive sample. FIG. 4A shows lateral flow assay strips that were run with an undiluted LAMP reaction amplifying nucleic acid from a contrived COVID positive sample. FIG. 4B shows lateral flow assay strips that were run with a diluted LAMP reaction amplifying nucleic acid from a contrived COVID positive sample.

FIGS. 5A-5D show images of exemplary lateral flow assay strips run for varying amounts of time with LAMP reactions amplifying nucleic acid from a COVID positive sample, where each lateral flow assay strip was tested for the pH of the fluid sample. Samples were applied to lateral flow assay strips and pH tested at 1 minute (FIG. 5A), 3 minutes (FIG. 5B), 6 minutes (FIG. 5C), or 9 minutes (FIG. 5D).

FIGS. 6A-6F show diagrams of labeled primers comprising exemplary extension blocking groups and/or haptens and depict removal or capture of said extension blocking groups and/or haptens.

FIG. 7 shows an image of three exemplary lateral flow assay strips comprising or not comprising an exemplary trap region comprising a plurality of positively-charged moieties and run with diluted or undiluted LAMP sample.

FIGS. 8A-8B show images of exemplary lateral flow assay strips comprising or not comprising an exemplary trap region comprising a plurality of positively-charged moieties in an exemplary first sub-region run with undiluted LAMP sample.

FIG. 9 shows an image of exemplary lateral flow assay strips comprising an exemplary trap region comprising a plurality of positively-charged moieties in an exemplary first sub-region. LAMP sample diluted to varying degrees (100% at right-most (undiluted) to 1.5% at left-most (an about 66 fold dilution)) was applied to the first sub-region of each lateral flow assay strip.

### DETAILED DESCRIPTION

The disclosure relates, in some aspects, to immunoassay devices for detecting target analytes (e.g., nucleic acids) in a sample and compositions and methods for detecting target analytes in said sample. The disclosure is based, in part, on compositions and methods that reduce the amount of one or more labeled primers and/or one or more labeled amplification products in an amplification reaction but still produce high contrast, visible bands when bound to certain immunoassay devices (e.g., lateral flow immunoassays). In some embodiments, immunoassay devices, compositions, and methods described herein advantageously allow for direct application of amplicons (e.g., solutions comprising amplicons) to immunoassay devices without any intervening fluid transfer or dilution steps.

### Biological Samples

Aspects of the disclosure relate to compositions and methods for detecting one or more target nucleotides in a biological sample. In some embodiments, a biological sample is obtained from a subject (e.g., a human subject, an animal subject). Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, a biological sample comprises blood, saliva, mucous, urine, feces, cerebrospinal fluid (CSF), or tissue. In some embodiments, the biological sample comprises a nasal secretion. In certain instances, for example, the sample is an anterior nares specimen. In some embodiments, the sample comprises an oral secretion (e.g., saliva). In some embodiments, a biological sample (e.g., saliva sample) is deposited directly into a reaction tube.

The biological sample, in some embodiments, is collected from a subject who is suspected of having the disease(s) the test screens for, such as a coronavirus (e.g., COVID-19) and/or influenza (e.g., influenza type A or influenza type B). A subject may be any mammal, for example a human, non-human primate (e.g., monkey, chimpanzee, ape, etc.), dog, cat, pig, horse, hamster, guinea pig, rat, mouse, etc. In some embodiments, a subject is a human.

### Nucleic Acids

The disclosure relates, in some aspects, to methods and systems for detecting nucleic acids and nucleic acid sequences. A “nucleic acid” sequence refers to a DNA or RNA sequence (or a sequence encoded by DNA or RNA). In some embodiments, a nucleic acid is isolated. As used herein, the term “isolated” means artificially produced. As used herein, with respect to nucleic acids, the term “isolated” means: (i) amplified in vitro by, for example, polymerase chain reaction (PCR) or isothermal nucleic acid amplification; (ii) recombinantly produced by cloning; (iii) purified, as by cleavage and gel separation; or (iv) synthesized by, for example, chemical synthesis. An isolated nucleic acid is one which can be manipulated by recombinant DNA techniques well known in the art. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

In some embodiments, a nucleic acid or isolated nucleic acid is a referred to as a “polynucleotide” or “oligonucleotide”. The terms “polynucleotide” and “oligonucleotide” refer to nucleic acids comprising two or more units (e.g., nucleotides) connected by a phosphate-based backbone (e.g., a sugar-phosphate backbone), for example genomic DNA (gDNA), complementary DNA (cDNA), RNA (e.g., mRNA, shRNA, dsRNA, miRNA, tRNA, etc.), synthetic nucleic acids and synthetic nucleic acid analogs. Polynucleotides (or oligonucleotides) may include natural or non-natural bases, or combinations thereof and natural or non-natural backbone linkages, such as phosphorothioate linkages, peptide nucleic acids (PNA), 2′-O-methyl-RNA, or combinations thereof.

The length of a polynucleotide (or each strand of a double stranded or duplex molecule) may vary. In some embodiments, a polynucleotide ranges from about 2 to 10, 2 to 20, 2 to 30, 2 to 40, 2 to 50, 2 to 75, 2 to 100, 2 to 150, 2 to 200, 2 to 300, 2 to 400, 2 to 500, 2 to 1000, 2 to 2000, 2 to 5000, 2 to 10,000, 2 to 50,000, 2 to 500,000, or 2 to 1,000,000 nucleotides in length. In some embodiments, a polynucleotide is more than 1,000,000 nucleotides in length (e.g., longer than a megabase).

A nucleic acid (e.g., a polynucleotide) may be single stranded or double stranded.

In some embodiments, a nucleic acid (e.g., polynucleotide) is a primer. As used herein, a “primer” refers to a polynucleotide that is capable of selectively binding (e.g., hybridizing or annealing) to a nucleic acid template and allows the synthesis of a sequence complementary to the corresponding polynucleotide template. A nucleic acid template may be a target nucleic acid or control nucleic acid. In some embodiments, a nucleic acid template comprises a region of complementarity with one or more primers. Generally, a primer ranges in size from about 10 to 100 nucleotides, and functions as a point of initiation for template-directed, polymerase mediated synthesis of a polynucleotide complementary to the template. A nucleic acid may be unmodified or modified. A modified nucleotide may comprise one or more modified nucleic acid bases and/or a modified nucleic acid backbone. In some embodiments, a modified nucleic acid comprises one or more nucleotide analogs.

In some embodiments, a nucleic acid is modified by conjugation to one or more biological or chemical moieties. Examples of moieties used for modifying nucleic acids include fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof. In some embodiments, a nucleic acid (e.g., a primer) comprises one or more modifications. In some embodiments, a modified nucleic acid comprises a hapten. In some embodiments, the hapten is FITC. In some embodiments, the hapten is digoxigenin (DIG). In some embodiments, the hapten is biotin. In some embodiments, a modified nucleic acid comprises an extension blocking group. Examples of extension blocking groups include but are not limited to dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, a modified nucleic acid comprises both a hapten and an extension blocking group.

### Target Nucleic Acids

Methods and compositions described by the disclosure may be used, in some embodiments, to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In certain embodiments, a diagnostic device comprises at lateral flow assay strip comprising a test line configured to detect a nucleic acid sequence of SARS-CoV-2. In certain embodiments, a diagnostic device comprises at lateral flow assay strip comprising a test line configured to detect a nucleic acid sequence of a SARS-CoV-2 virus having one or more mutations. In some embodiments, the one or more mutations comprise a D614G mutation (i.e., a mutation of the 614^{th} amino acid from aspartic acid (D) to glycine (G)) in its spike protein. In some embodiments, the one or more mutations comprise 1, 2, 3, 4, 5 or all of D614G, A222V, N501Y, E484K, K417N, K417T, S494P, A570D, P681H, T716I, S982A, D1118H, K1191N, D80A, D215G, A701V, T19R, V70F, T95I, G142D, R158G, W258L, L452R, T478K, P681R, D950N, L18F, T20N, P26S, D138Y, R190S, H655Y, T1027I, S131, W152C, LSF, D80G, F157S, D253G, S477N, T859N, D950H, Q957R, E154K, E484Q, and/or Q1071H mutations. In some embodiments, the SARS-CoV-2 virus having one or more mutations is SARS-CoV-2 D614G, a SARS-CoV-2 variant of B.1.1.7 lineage (e.g., 201/501Y.V1 Variant of Concern (VOC) 202012/01), a SARS-CoV-2 variant of B.1.351 lineage (e.g., 20H/501.V2), a SARS-CoV-2 variant of B.1.617.2 lineage (e.g., 21A/S:478K), a SARS-CoV-2 variant of P.1 lineage (e.g., 20J/501Y.V3), a SARS-CoV-2 variant of B.1.427 lineage (e.g., 20C/S:452R), a SARS-CoV-2 variant of B.1.429 lineage (e.g., 20C/S:452R), a SARS-CoV-2 variant of B.1.525 lineage (e.g., 20A/S:484K), a SARS-CoV-2 variant of B.1.526 lineage (e.g., 20C/S:484K), a SARS-CoV-2 variant of B.1.617.1 lineage (e.g., 21A/S:154K), or a SARS-CoV-2 variant of B.1.617.3 lineage (e.g., 20A). In certain embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having one or more mutations. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to diagnose two or more diseases or disorders. In certain cases, for example, a diagnostic device comprises a lateral flow assay strip comprising a first test line configured to detect a nucleic acid sequence of SARS-CoV-2 and a second test line configured to detect a nucleic acid sequence of an influenza virus (e.g., an influenza A virus or an influenza B virus). In some embodiments, a diagnostic device comprises a lateral flow assay strip comprising a first test line configured to detect a nucleic acid sequence of a virus and a second test line configured to detect a nucleic acid sequence of a bacterium. In some embodiments, a diagnostic device comprises a lateral flow assay strip comprising three or more test lines (e.g., test lines configured to detect SARS-CoV-2, a SARS-CoV-2 virus having one or more mutations (e.g., D614G), an influenza type A virus, and/or an influenza type B virus). In some embodiments, a diagnostic device comprises a lateral flow assay strip comprising four or more test lines (e.g., test lines configured to detect SARS-CoV-2, a SARS-CoV-2 virus having one or more mutations (e.g., D614G), an influenza type A virus, and/or an influenza type B virus).

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2 and/or a SARS-CoV-2 virus having one or more mutations (e.g., described herein, e.g., D614G). In certain embodiments, the viral pathogen is an influenza virus. The influenza virus may be an influenza A virus (e.g., H1N1, H3N2) or an influenza B virus.

In some embodiments, the diagnostic devices, systems, and methods are configured to detect a target nucleic acid sequence of a bacterium (e.g., a bacterial pathogen). In some embodiments, the diagnostic devices, systems, and methods are configured to detect a target nucleic acid sequence of a fungus (e.g., a fungal pathogen). In some embodiments, the diagnostic devices, systems, and methods are configured to detect a target nucleic acid sequence of one or more protozoa (e.g., a protozoan pathogen).

In some embodiments, the diagnostic devices, systems, and methods are configured to detect a target nucleic acid sequence of a cancer cell. Cancer cells have unique mutations found in tumor cells and absent in normal cells. In some embodiments, the diagnostic devices, systems, and methods are configured to examine a subject's predisposition to certain types of cancer based on specific genetic mutations. In some embodiments, the diagnostic devices, systems, and methods are configured to detect a target nucleic acid sequence associated with a genetic disorder.

In some embodiments, the diagnostic devices, systems, and methods are configured to detect a target nucleic acid sequence of an animal pathogen.

### Control Nucleic Acid Sequences

In some embodiments, methods and systems described herein comprise (or use) primers designed to amplify a human or animal nucleic acid that is not associated with a target nucleic acid from a pathogen, a cancer cell, or a contaminant. In some such embodiments, the human or animal nucleic acid may act as a control and is referred to as a “control nucleic acid”. For example, successful amplification and detection of a control nucleic acid may indicate that a sample was properly collected, and the diagnostic test was properly run (e.g., an amplification reaction was successful).

A control nucleic acid is typically a gene or portion of a gene that is widely expressed and/or expressed at a high level in a control organism (e.g., a human or other mammal). Such genes are typically referred to as “housekeeping genes”. Examples of housekeeping genes include but are not limited to RNase P, GAPDH, B2M, ACTB, POLR2A, UBC, PPIA, HPRT1, GUSB, TBP, and H3F3A. In some embodiments, a control nucleic acid encodes a gene (or portion of a gene) selected from GAPDH, B2M, ACTB, POLR2A, UBC, PPIA, HPRT1, GUSB, TBP, H3F3A, POLR2A, RPLPO, L19, B2M, RPS17, ALAS1, CD74, RNase P, CK18, HMBS, IPO8, PGK1, YWHAZ, and STATH.

### Lysis of Sample

A biological sample may be lysed prior to (or while) performing an amplification reaction (e.g., an isothermal amplification reaction, such as RT-LAMP). In some embodiments, lysis of a biological sample is performed by chemical lysis (e.g., exposing a sample to one or more lysis reagents) and/or thermal lysis (e.g., heating a sample). Chemical lysis may be performed by one or more lysis reagents. In some embodiments, the one or more lysis reagents comprise one or more enzymes. Non-limiting examples of suitable enzymes include lysozyme, lysostaphin, zymolase, cellulase, protease, and glycanase.

In some embodiments, the one or more lysis reagents comprise one or more detergents. In some embodiments, an amplification buffer described herein comprises one or more detergents. In some embodiments, the one or more detergents comprise Tween.

In some cases, at least one of the one or more lysis reagents is in solid form (e.g., lyophilized, dried, crystallized, air jetted).

In some embodiments, cell lysis is accomplished by applying heat to a sample (thermal lysis). In some embodiments, a lysis heating protocol may comprise heating a sample at one or more additional temperatures for one or more additional time periods.

### Methods of Amplifying Nucleic Acids

Aspects of the disclosure relate to methods and compositions for detecting target nucleic acids and/or control nucleic acids in a biological sample. In some embodiments, the methods comprise a step of performing an isothermal amplification reaction on a biological sample in order to amplify a target nucleic acid and/or a control nucleic acid.

In some embodiments, performing an isothermal amplification reaction on a biological sample comprising a target nucleic acid generates labeled target amplification products. In some embodiments, performing an isothermal amplification reaction on a biological sample comprising a control nucleic acid generates labeled control amplification products. In some embodiments, after performing an isothermal amplification reaction, a biological sample or a resulting fluid sample comprises labeled unextended primers or unlabeled primers. In some embodiments, performing an isothermal amplification reaction on a biological sample produces one or more unintended side products, e.g., labeled or unlabeled extended primer hairpins, primer dimers, or non-specific amplification products not comprising target or control nucleic acid.

The disclosure is based, in part, on amplification buffer solutions comprising one or more of the following components: biotinylated oligonucleotides, a target nucleic acid-specific primer comprising a first hapten, and a control nucleic acid-specific primer comprising a second hapten different from the first hapten. In some embodiments, the buffer comprises a total concentration of biotinylated oligonucleotides that is less than 200 nM, a total concentration of first hapten less than or equal to 25 nM, and a total concentration of second hapten that is less than or equal to 100 nM. In some embodiments, the result of performing an amplification reaction using such a buffer is production of a mixture of amplification products that can be directly added to a detection device, for example a lateral flow assay (LFA) device, and which results in visible bands. This is surprising because it was previously thought that amplification mixtures needed to be diluted prior to addition to a detection device in order to produce brightly visible bands.

Thus, in some embodiments, following lysis, one or more target nucleic acids (e.g., a nucleic acid of a target pathogen) and/or one or more control nucleic acids may be amplified. In some cases, a target pathogen has RNA as its genetic material. In certain instances, for example, a target pathogen is an RNA virus (e.g., a coronavirus, an influenza virus). In some such cases, the target pathogen's RNA may need to be reverse transcribed to DNA prior to amplification.

In some embodiments, reverse transcription is performed by exposing lysate (or nucleic acids within a lysate) to one or more reverse transcription reagents. In certain instances, the one or more reverse transcription reagents comprise a reverse transcriptase, a DNA-dependent polymerase, and/or a ribonuclease (RNase). A reverse transcriptase generally refers to an enzyme that transcribes RNA to complementary DNA (cDNA) by polymerizing deoxyribonucleotide triphosphates (dNTPs). An RNase generally refers to an enzyme that catalyzes the degradation of RNA. In some cases, an RNase may be used to digest RNA from an RNA-DNA hybrid.

In some embodiments, a reverse transcriptase used for an isothermal amplification reaction (e.g., RT-LAMP) is selected from the group consisting of HIV-1 reverse transcriptase, Moloney murine leukemia virus (M-MLV) reverse transcriptase, and avian myeloblastosis virus (AMV) reverse transcriptase.

In some embodiments, DNA may be amplified according to any nucleic acid amplification method known in the art. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP).

In some cases, at least one of the one or more amplification reagents is in solid form (e.g., lyophilized, dried, crystallized, air jetted). In some cases, all of the one or more amplification reagents are in solid form (e.g., lyophilized, dried, crystallized, air jetted). In certain embodiments, one or more amplification reagents are in the form of an amplification pellet or tablet. The amplification pellet or tablet may comprise any amplification reagent described herein.

In some embodiments, one, two, or all of the biotinylated oligonucleotides, the target nucleic acid-specific primer comprising a first hapten, or the control nucleic acid-specific primer comprising a second hapten different from the first hapten further comprise an extension blocking group. As used herein, an extension blocking group refers to a moiety that prevents a polymerase from adding nucleotides to the 3′ end of a nucleic acid such as an oligonucleotide or primer. In some embodiments, the extension blocking group is positioned at the 3′ end of the nucleic acid (e.g., oligonucleotide or primer). In some embodiments, an extension blocking group comprises a dehydroxylated 3′ nucleotide, 3′ dideoxycytosine (ddC), 3′ inverted deoxythymidine (dT), 3′ propyl (C3) spacer, 3′ amino modification, or 3′ phosphoryl modification. In some embodiments, a polymerase used in a method of amplifying nucleic acids (e.g., described herein) comprises an activity capable of removing the extension blocking group.

In some embodiments, a polymerase comprising an exonuclease activity is capable of removing the extension blocking group. In some embodiments, a polymerase used in a method of amplifying nucleic acids (e.g., described herein) does not comprise an activity capable of removing the extension blocking group, and a separate enzyme (e.g., an exonuclease) capable of removing the extension blocking group is included in the amplification reaction.

### LAMP

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence. In some embodiments, LAMP is combined with a reverse transcription reaction, and referred to as RT-LAMP.

In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample. In some embodiments, the LAMP reagents comprise LAMP primers designed to amplify a human or animal nucleic acid that is not associated with a pathogen, a cancer cell, or a contaminant (e.g., a control nucleic acid). In some such embodiments, the human or animal nucleic acid may act as a control. For example, successful amplification and detection of the control nucleic acid may indicate that a sample was properly collected, and the diagnostic test was properly run.

In some embodiments, one or more LAMP primers comprise a label. In some embodiments, a label refers to a moiety that is attached to a primer and facilitates visualization of the primer or its extension product. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay). In certain embodiments, the label is a fluorescent label. In some instances, the fluorescent label is associated with a quenching moiety that prevents the fluorescent label from signaling until the quenching moiety is removed. In certain embodiments, a LAMP primer is labeled with two or more labels. In some embodiments, one or more LAMP primers comprise a hapten. In some embodiments, a hapten refers to a moiety that may be attached to a primer and used as a target to specifically bind the primer or its extension product. Non-limiting examples of suitable haptens include biotin, streptavidin, fluorescein isothiocyanate (FITC), fluorescein amidite (FAM), fluorescein, and digoxigenin (DIG). The distinction of whether a moiety attached to a labeled primer or labeled product is a label or a hapten is one of function. For example, in some embodiments, a first exemplary labeled primer comprises FITC and a second exemplary labeled primer comprises biotin, and both are used in an exemplary amplification reaction to produce an amplification product mixture which is applied to a lateral flow assay strip. The exemplary lateral flow assay strip comprises a conjugate pad comprising streptavidin-gold particles and a test line comprising immobilized antibodies against FITC. In such embodiments, FITC may be referred to as a hapten because it is a moiety used as a target to specifically bind a primer extension product. In such embodiments, biotin may be referred to as a label, since it is a moiety for facilitating visualization of the primer extension product.

The disclosure is based, in part, on LAMP amplification buffers comprising biotinylated oligonucleotides (e.g., biotinylated primers). In some embodiments, a LAMP amplification buffer comprises one or more target nucleic acid-specific biotinylated primers. In some embodiments, a LAMP amplification buffer comprises one or more control nucleic acid-specific biotinylated primers. The biotinylated primers may be outer primers, inner primers and/or loop primers.

In some embodiments, a buffer solution further comprises one or more sets (e.g., pairs) of outer primers. In some embodiments, at least one set (e.g. pair) of outer primers specifically binds to a target nucleic acid. In some embodiments, at least one set of outer primers specifically binds to a control nucleic acid.

In some embodiments, a buffer solution further comprises one or more additional sets (e.g., pairs) of internal primers. In some embodiments, at least one set (e.g., pair) of internal primers specifically binds to a target nucleic acid. In some embodiments, at least one set (e.g., pair) of internal primers specifically binds to a control nucleic acid.

In some embodiments, an amplification buffer comprises a DNA polymerase with high strand displacement activity.

In some embodiments, the LAMP reagents comprise deoxyribonucleotide triphosphates (“dNTPs”). In some embodiments, the LAMP reagents comprise magnesium sulfate (MgSO₄). In some embodiments, the LAMP reagents comprise betaine.

In some embodiments, performing LAMP on a biological sample comprising a target nucleic acid generates labeled target amplification products. In some embodiments, performing LAMP on a biological sample comprising a control nucleic acid generates labeled control amplification products. In some embodiments, after performing LAMP, a biological sample or a resulting fluid sample comprises labeled unextended primers or unlabeled primers. In some embodiments, performing LAMP on a biological sample produces one or more unintended side products, e.g., labeled or unlabeled extended primer hairpins, primer dimers, or non-specific amplification products not comprising target or control nucleic acid.

### Oligonucleotide Strand Displacement Probes

Aspects of the disclosure relate to the recognition that inclusion of certain oligonucleotide strand displacement (OSD) probes in isothermal amplification reactions (e.g., LAMP) allows for direct application of the resulting amplicon mixtures to immunoassay devices without any intervening fluid transfer or dilution steps. An “oligonucleotide strand displacement probe” generally refers to a modified polynucleotide primer comprising a region of complementarity with one or more target nucleotides that is capable of displacing pre-hybridized strands of target nucleic acid amplicons (e.g., amplicons generated by LAMP). Oligonucleotide stand-displacement probes are generally known, for example as described by Jiang et al., Angew Chem Int Ed Engl. 2014 Feb. 10; 53(7): 1845-1848; Phillips et al., Anal Chem. 2018 Jun. 5; 90(11): 6580-6586; and Bhadra et al. (2020) https://doi.org/10.1101/2020.04.13.039941.

### Detection

In some embodiments, amplified nucleic acids (e.g., amplicons of target nucleic acids or control nucleic acids) may be detected using any suitable methods. In some embodiments, one or more target nucleic acid sequences are detected using a lateral flow assay strip. In some embodiments, one or more target nucleic acid sequences are detected using a colorimetric assay.

Aspects of the disclosure relate to methods that result in detection of one or more signals on a lateral flow assay that are brighter (e.g., more intense, more easily visible to an unaided eye, etc.) than lateral flow assay signals produced using previous methods. In some embodiments, signals (e.g., visible bands, e.g., colored bands) produced using methods of detecting a target nucleic acid described herein (e.g., using a lateral flow assay strip comprising a trap region or a method using a trap solution) are between 10% and 100% (10, 20, 30, 40, 50, 60, 70 80, 90, 95, 99, or 100%) brighter than lateral flow signals produced using previously available methods. In some embodiments, signals (e.g., colored bands) produced using a lateral flow assay strip described herein or methods of detecting a target nucleic acid described herein (e.g., using a lateral flow assay strip comprising a trap region or a method using a trap solution) are at least 10, 20, 30, 40, 50, 60, 70, 80, 90, 100, 200, or 300% more intense than signals that would be obtained in an otherwise similar device or method wherein the lateral flow assay strip does not comprise a trap region or the method does not use a trap solution. Aspects of the disclosure relate to methods of detecting a target nucleic acid (e.g., on a lateral flow assay strip, e.g., described herein) that produce fewer false negative results than previous methods, e.g., methods using lateral flow assay strips not comprising a trap region. In some embodiments, a method of detecting a target nucleic acid described herein (e.g., using a lateral flow assay strip comprising a trap region or a method using a trap solution) produces at least 10, 20, 30, 40, 50, 60, 70, 80, 90, or 100% fewer false negative results than an otherwise similar method wherein the lateral flow assay strip does not comprise a trap region or not using a trap solution.

The high dose effect refers to a decrease in the formation of immune complexes in immunoassays at high concentrations of either antigen or antibody. This phenomenon has previously been observed in methods of detection utilizing lateral flow assay strips. Sample dilution has been suggested as a means to mitigate the decrease in immune complex formation and thereby increase assay signal. However, sample dilution steps can be cumbersome and exceed the technical capabilities of users of at-home or point-of-care devices and methods, where precise fluid transfer mechanisms may not be available or introduce the risk of error. The disclosure is directed, in part, to the discovery that the level of nucleic acid, e.g., of one, two, three, or all of labeled target amplification products, labeled control amplification products, labeled unextended primers, or unlabeled primers, in a fluid sample can be decreased without a sample dilution step (e.g., an amplified sample may be applied directly to an immunoassay device (e.g., a lateral flow assay strip)). Without wishing to be bound by theory, an immunoassay device comprising a trap region, e.g., comprising one or more trap reagents, can be used to decrease the level of one, two, three, or all of labeled target amplification products, labeled control amplification products, labeled unextended primers, or unlabeled primers in a fluid sample. In some embodiments, a trap region, e.g., comprising one or more trap reagents, can be used to decrease the level of unintended side products of an isothermal amplification process (e.g., LAMP), e.g., labeled or unlabeled extended primer hairpins, primer dimers, or non-specific amplification products not comprising target or control nucleic acid. In some embodiments, the trap region comprises a plurality of nucleic acid binding moieties capable of binding nucleic acid non-specifically (i.e., in a sequence non-specific manner), e.g., a plurality of positively charged moieties. In some embodiments, the trap region comprises one or more trap reagents that specifically bind to one, two, three, or all of labeled target amplification products, labeled control amplification products, labeled unextended primers, or unlabeled primers, decreasing the level of said labeled products or primers in the fluid sample. In some embodiments, the trap region comprises one or more trap reagents that specifically bind to unintended side products of an isothermal amplification process (e.g., LAMP), e.g., labeled or unlabeled extended primer hairpins, primer dimers, or non-specific amplification products not comprising target or control nucleic acid, decreasing the level of said unintended side products in the fluid sample. A fluid sample comprising said decreased level of nucleic acid may subsequently flow to a sub-region comprising one or more test lines, resulting in detection of one or more signals on the lateral flow assay strip that are brighter (e.g., more intense, more easily visible to an unaided eye, etc.) than lateral flow assay signals produced using previous methods (e.g., not employing a trap region, e.g., comprising one or more trap reagents).

Accordingly, in some embodiments, a method described herein does not comprise a dilution step (e.g., does not comprise diluting a fluid sample or an amplification product mixture). In some embodiments, the fluid sample, amplification product mixture, or aliquot thereof used in a method described herein or applicable to a device described herein is undiluted. As used herein, describing a sample (e.g., a fluid sample or amplification product mixture) as undiluted or not having been diluted means that the level of one or more components of the sample has not been decreased by addition of a diluent. In some embodiments, a decrease (e.g., the lack of decrease) is relative to a reference level of the one or more components (e.g., where the reference level is the level of the one or more components at the completion of a previous method step, e.g., generation of a fluid sample or amplification product mixture). In some embodiments, the reference level of an amplification product mixture component (e.g., a labeled target amplification product, a labeled control amplification product, or a labeled unextended primer) is the level of said component present at the time the amplification product mixture was generated (e.g., the time at which an isothermal amplification process completed, generating the amplification product mixture). The disclosure is directed, in part, to improved methods and devices which can decrease the level of one or more sample components (e.g., a labeled target amplification product, a labeled control amplification product, a labeled unextended primer, or unintended side products of an isothermal amplification process (e.g., LAMP)) without a dilution step.

In some embodiments, one or more target nucleic acid sequences are detected using a lateral flow assay strip (e.g., in a “chimney” detection component, a cartridge, a blister pack). In some embodiments, a fluid sample (e.g., fluidic contents of a reaction tube, a reagent reservoir, and/or a blister pack chamber) is transported through the lateral flow assay strip via capillary action. In some embodiments, the fluid sample may comprise labeled amplicons.

In some embodiments, the fluid sample is introduced to a first sub-region (e.g., a sample pad) of the lateral flow assay strip. In certain embodiments, the fluid sample subsequently flows through a second sub-region (e.g., a particle conjugate pad) comprising a plurality of labeled particles. In some cases, the particles comprise gold nanoparticles (e.g., colloidal gold nanoparticles), latex particles (e.g., colored latex beads), silver particles (e.g., silver nanoparticles), magnetic particles, quantum dots, and/or carbon particles (e.g., carbon nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluid sample flows through the second sub-region (e.g., a particle conjugate pad), a labeled nanoparticle binds to a label moiety of an amplicon, thereby forming a particle-amplicon conjugate.

In some embodiments, the fluid sample (e.g., comprising a particle-amplicon conjugate) subsequently flows through a third sub-region (e.g., a test pad) comprising one or more test lines. In some embodiments, a first test line comprises a capture reagent (e.g., an immobilized antibody) configured to detect a first target nucleic acid. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay strip further comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip. In certain instances, a first control line is a lateral flow control line. In some cases, the lateral flow control line becoming detectable indicates that a liquid was successfully transported through the lateral flow assay strip. In some embodiments, the lateral flow assay strip comprises two or more control lines. The control line(s) may have any suitable shape or pattern (e.g., one or more straight lines, curved lines, dots, squares, check marks, x marks). In some instances, for example, the lateral flow assay strip comprises a human (or animal) nucleic acid control line and a lateral flow control line.

In certain embodiments, the lateral flow assay strip comprises a first sub-region wherein the fluid sample can be introduced to the lateral flow assay strip. In some embodiments, the first sub-region comprises a trap region (e.g., comprising a trap pad). In some embodiments, the trap region comprises a trap pad. In some embodiments, a trap pad comprises an absorbent substrate that is separate from the material comprising another sub-region (e.g., the third sub-region comprising one or more test lines), while remaining in fluidic communication with other sub-regions of the lateral flow assay strip. In some embodiments, the trap pad is part of the absorbent substrate of at least one other sub-region (e.g., the second sub-region, the third sub-region, the fourth sub-region, or all of the other sub-regions). In some embodiments, a trap pad comprises a plurality of nucleic acid binding moieties, e.g., a plurality of positively charged moieties. In some embodiments, a trap pad comprises a trap reagent immobilized to the absorbent substrate.

In certain embodiments, the lateral flow assay strip comprises a fourth sub-region comprising a trap region (e.g., comprising a trap pad or one or more trap lines). In some embodiments, the trap region comprises a plurality of nucleic acid binding moieties, e.g., a plurality of positively charged moieties. In some embodiments, the trap region comprises one or more trap reagents. In some embodiments, the trap region comprises a trap pad. In some embodiments, a trap pad comprises an absorbent substrate that is separate from the material comprising another sub-region (e.g., the third sub-region comprising one or more test lines), while remaining in fluidic communication with other sub-regions of the lateral flow assay strip. In some embodiments, a trap pad comprises a trap reagent or plurality of positively charged moieties immobilized to the absorbent substrate. In some embodiments, the trap region comprises one or more trap lines. In some embodiments, the one or more trap lines comprise trap reagents or a plurality of positively charged moieties immobilized in one or more lines on the same material (e.g., the same absorbent substrate) as another sub-region (e.g., the third sub-region comprising one or more test lines).

Nucleic Acid Binding Moieties

In some embodiments, the trap region comprises a plurality of nucleic acid binding moieties. In some embodiments, the plurality of nucleic acid binding moieties is capable of binding nucleic acid non-specifically (i.e., in a sequence non-specific manner). In some embodiments, the plurality of nucleic acid binding moieties is or comprises a plurality of positively charged moieties. In some embodiments, the plurality of nucleic acid binding moieties comprises a single type of nucleic acid binding moieties. In some embodiments, the plurality of nucleic acid moieties comprises multiple types of nucleic acid binding moieties (e.g., a first type of nucleic acid binding moieties and a second type of nucleic acid binding moieties). Types of nucleic acid binding moieties may be distinguished from one another by chemical identity (e.g., a plurality of nucleic acid binding moieties comprising a first type comprising trimethylammonium moieties and a second type comprising DEAE moieties) and/or by binding specificity (e.g., a plurality of nucleic acid binding moieties comprising a first type comprising trimethylammonium moieties exhibiting sequence non-specific nucleic acid binding and a second type comprising an oligonucleotide complementary to a target nucleic acid).

In some embodiments, the nucleic acid binding moieties are or comprise positively charged moieties. As used herein, a positively charged moiety refers to a chemical entity that is positively charged at a reference pH. In some embodiments, the reference pH is the pH of a fluid sample (e.g., a fluid sample for use in a method or device described herein). In some embodiments, the reference pH is the pH of an amplification product mixture or the pH at which an amplification reaction that produced an amplification product mixture occurred. In some embodiments, the reference pH is 2-8, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8, e.g., pH 3, 4, 5, 6, 7, or 8. In some embodiments, a positively charged moiety has a pKa of 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14, e.g., a pKa of 8-12, e.g., a pKa of approximately 8, 9, 10, or 11. Without wishing to be bound by theory, the difference between the pKa of a chemical entity and the surrounding pH determines the extent to which a chemical entity is protonated, e.g., positively charged. For example, since pH and pKa are log scale parameters, 90% of a population of chemical entities with a pKa of 8 in a solution at pH 7 will be protonated and 10% will be unprotonated. If the solution pH drops to 6, 99% of the entities will be protonated and 1% will be unprotonated. In some embodiments, referring to a positively charged moiety as “positively charged at a reference pH” means that in a group of the positively charged moieties at least 50, 90, 95, 99, 99.5, 99.9, 99.95, 99.99, 99.995, or 99.999% of the moieties are positively charged at a given time at the reference pH. In some embodiments, the pH of a fluid sample (e.g., for use in a method or device described herein) is selected to ensure the plurality of positively charged moieties is positively charged (e.g., at least 50, 90, 95, 99, 99.5, 99.9, 99.95, 99.99, 99.995, or 99.999% of the moieties are positively charged) when interacting with the fluid sample. For example, a trap region may comprise a plurality of positively charged moieties comprising TRIS (tris(hydroxymethyl)aminomethane), which has a pKa (isolated in solution) of approximately 8. The pH of a fluid sample may be chosen (e.g., adjusted) to be 8 or lower (e.g., less than 8, 7.5, 7, 6.5, or 6) to ensure that the positively charged moieties of the trap region are positively charged (e.g., at least 50, 90, 95, 99, 99.5, 99.9, 99.95, 99.99, 99.995, or 99.999% of the moieties are positively charged) when interacting with the fluid sample.

In some embodiments, a positively charged moiety is attached or attachable to another molecule or structure (e.g., a polymer, e.g., a polysaccharide), e.g., making up an absorbent substrate. The positively charged moiety may be attached to another molecule or structure by one or more covalent bonds, non-covalent interactions (e.g., hydrogen bonds, electrostatic interactions), and/or physical associations. Without wishing to be bound by theory, a plurality of positively charged moieties attached to a structure (e.g., a polymer, e.g., a polysaccharide) may interact with one another and/or elements of the structure in a manner that alters one or more of their properties, e.g., the pKa of one or more of the positively charged moieties. For example, adjacent ionic interactions of the positively charged moieties attached to a polymer may cause the positively charged moieties to exhibit a range of pKa values. In some embodiments, a trap region comprises a plurality of positively charged moieties (e.g., attached to a structure (e.g., a polymer, e.g., a polysaccharide) that exhibit pKa values from 6-14, 6-13, 6-12, 6-11, 6-10, 6-9, 6-8, 6-7, 7-14, 7-13, 7-12, 7-11, 7-10, 7-9, 7-8, 8-14, 8-13, 8-12, 8-11, 8-10, 8-9, 9-14, 9-13, 9-12, 9-11, 9-10, 10-14, 10-13, 10-12, 10-11, 11-14, 11-13, 11-12, 12-14, 12-13, or 13-14, e.g., pKa values of 8-12. A person of skill in the art will recognize that, while the pKa of an isolated positively charged moiety or a plurality of positively charged moieties situated in relatively similar molecular surroundings may be measured without undue difficulty and expected to be approximately uniform, the pKa of a plurality of positively charged moieties interacting with one another and/or different elements of a structure to which they are attached (e.g., a polymer, e.g., a polysaccharide), e.g., an absorbent substrate, may encompass a range of values.

Without wishing to be bound by theory, positively charged moieties are capable of binding nucleic acid non-specifically (e.g., by electrostatically interacting with the negatively charged phosphate backbone of a nucleic acid), decreasing the level of nucleic acid (e.g., one, two, three, four, or all of the labeled target amplification products, labeled control amplification products, labeled unextended primers, unlabeled primers, or unintended side products of an isothermal amplification process (e.g., LAMP)) in a fluid sample. By decreasing the level of nucleic acid (e.g., all nucleic acids) in a fluid sample, a trap region comprising a plurality of positively charged moieties may allow a lateral flow assay strip or immunoassay method employing the same to produce high contrast, visible bands, e.g., without any dilution steps or intervening fluid manipulation steps between an amplification reaction and application to the lateral flow assay strip. In some embodiments, the decrease in the level of nucleic acid is sufficient to produce high contrast, visible bands without decreasing the level of nucleic acid to the extent that detection is impaired (e.g., without completely depleting a fluid sample of target nucleic acid or labeled amplification products).

In some embodiments, the decrease in the level of nucleic acid achieved by a trap region comprising a plurality of positively charged moieties is modulated and/or tuned by adjusting the nucleic acid binding capacity of the trap region. In some embodiments, the nucleic acid binding capacity of the trap region is represented by the ionic exchange capacity of the trap region (e.g., in mEq/g). In some embodiments, the nucleic acid binding capacity or ionic exchange capacity is less than the amount of nucleic acid (e.g., the amount of one, two, three, or all of the labeled target amplification products, labeled control amplification products, labeled unextended primers, or unlabeled primers) in the fluid sample. In some embodiments, the nucleic acid binding capacity of the trap region is represented by the density or concentration (e.g., molar concentration) of positively charged moieties in the trap region. In some embodiments, the density or concentration of positively charged moieties in the trap region is less than the amount (e.g., molar equivalents) of nucleic acid (e.g., the amount of one, two, three, or all of the labeled target amplification products, labeled control amplification products, labeled unextended primers, or unlabeled primers) in the fluid sample. In some embodiments, the degree of substitution of the absorbent substrate (e.g., the hydroxyl groups of the absorbent substrate) corresponds to the nucleic acid binding capacity of the trap region and is adjusted to be less than the amount (e.g., molar equivalents) of nucleic acid (e.g., the amount of one, two, three, or all of the labeled target amplification products, labeled control amplification products, labeled unextended primers, or unlabeled primers) in the fluid sample. In some embodiments, the degree of substitution of the polymer (e.g., polysaccharide, acrylic polymer, or styrenic polymer) is at least 0.1, 0.5, 1, 2, 3, 4, 5, 6, 7, 8, 9, 10, 12, 14, 16, 18, or 20%, or 0.1-20, 1-20, 2-20, 4-20, 6-20, 8-20, 10-20, 12-20, 14-20, 16-20, 18-20, 0.1-18, 1-18, 2-18, 4-18, 6-18, 8-18, 10-18, 12-18, 14-18, 16-18, 0.1-16, 1-16, 2-18, 4-16, 6-16, 8-16, 10-16, 12-16, 14-16, 0.1-14, 1-14, 2-14, 4-14, 6-14, 8-14, 10-14, 12-14, 0.1-12, 1-12, 2-12, 4-12, 6-12, 8-12, 10-12, 0.1-10, 1-10, 2-10, 4-10, 6-10, 8-10, 0.1-8, 1-8, 2-8, 4-8, 6-8, 0.1-6, 1-6, 2-6, 4-6, 0.1-4, 1-4, 2-4, 0.1-2, 1-2, or 0.1-1%.

A person of skill in the art will understand that, where a lateral flow assay strip, device containing the same, or method employing the aforementioned is intended for use with a fluid sample of unknown nucleic acid concentration, estimates and average expected nucleic acid levels may guide modulation of desired nucleic acid binding capacity of a trap region (e.g., comprising a plurality of positively charged moieties). Without wishing to be bound by theory, to improve the signal of a lateral flow assay strip a trap region can decrease the level of nucleic acid (e.g., the amount of one, two, three, or all of the labeled target amplification products, labeled control amplification products, labeled unextended primers, or unlabeled primers) in the fluid sample but binding 100% of the nucleic acid in the fluid sample would eliminate signal entirely.

In some embodiments, the decrease in the level of nucleic acid achieved by a trap region comprising a plurality of positively charged moieties is modulated and/or tuned by adjusting the size (e.g., the area (e.g., surface area) or volume) of the trap region, thereby modulating and/or tuning nucleic acid binding capacity of the trap region. Without wishing to be bound by theory, a fluid sample that traverses a larger volume or surface area of a trap region comprising a plurality of positively charged moieties at a given density would have a greater decrease in the level of nucleic acid upon exiting the trap region than a fluid sample that traversed an otherwise similar trap region with a smaller volume or surface area. For example, in some embodiments, a lateral flow assay strip comprising a longer trap region (or method utilizing the same) is capable of producing a visible signal from a sample containing a higher concentration of analyte than an otherwise similar lateral assay flow strip comprising a shorter trap region. As used herein, “length” in reference to a trap region refers to the dimension of the trap region that is parallel with the direction of flow of a sample. In some embodiments, the length of a trap region or first sub-region (e.g., the absorbent substrate of the first sub-region) comprising a trap region is selected to modulate and/or tune the nucleic acid binding capacity of the trap region. In some embodiments, the length of a trap region or first sub-region (e.g., the absorbent substrate of the first sub-region) comprising a trap region is 10-30, 10-25, 10-24, 10-23, 10-22, 10-21, 10-20, 10-19, 10-18, 10-17, 10-16, 10-15, 10-14, 10-13, 10-12, 10-11, 15-30, 15-25, 15-24, 15-23, 15-22, 15-21, 15-20, 15-19, 15-18, 15-17, or 15-16 millimeters (mm). In some embodiments, the width of a trap region or first sub-region (e.g., the absorbent substrate of the first sub-region) comprising a trap region is approximately 3, approximately 4, approximately 5, approximately 6, or approximately 7 mm (e.g., 3-7 or 4-6 mm). As used herein, “width” in reference to a trap region refers to a dimension of the trap region that is perpendicular to the direction of flow of a sample and in the same horizontal plane as the length dimension. In some embodiments, a trap region or first sub-region (e.g., the absorbent substrate of the first sub-region) comprising a trap region is approximately 5 mm wide and approximately 10-25 mm long, or approximately 5 mm wide and approximately 15-20 mm long.

In some embodiments, the thickness of a trap region or first sub-region (e.g., the absorbent substrate of the first sub-region) comprising a trap region is selected to modulate and/or tune the nucleic acid binding capacity of the trap region. In some embodiments, the thickness of a trap region (or sub-region comprising the trap region) is greater than the thickness of one or more adjacent sub-region of a lateral flow assay strip.

In some embodiments, the dimensions (e.g., length) of the trap region or first sub-region (e.g., the absorbent substrate of the first sub-region) comprising a trap region are selected such that a lateral flow assay strip comprising the trap region or first-subregion comprising the trap region (or method using the same) has a suitable detection range. As used herein, “detection range” refers to the range of analyte concentrations in a sample applied to an assay or device (e.g., a lateral flow assay strip) which produces a detectable signal in the assay or device (e.g., a visible signal on a lateral flow assay strip). In some embodiments, the detection range of a lateral flow assay strip comprising a trap region described herein (or a method using the same) encompasses at least an approximately 10, 20, 30, 40, 50, 60, 70, 80, 90, 100, 110, 120, 130, 140, 150, or 200 fold range of analyte concentrations (wherein a 100 fold range of concentrations refers to an upper range boundary that is 100 times larger than a lower range boundary). In some embodiments, one or more dimensions (e.g., length) of the trap region or first sub-region (e.g., the absorbent substrate of the first sub-region) comprising a trap region are altered (e.g., the length is increased) responsive to a previous lateral flow assay strip or method where a sample did not produce a detectable signal. In some embodiments, a method described herein further comprises, responsive to not observing a detectable signal in a previous lateral flow assay strip or method step, selecting or altering the length of a trap region or first sub-region (e.g., the absorbent substrate of the first sub-region) comprising a trap region of a further lateral flow assay strip, and analyzing a previously-run sample on the further lateral flow assay strip.

In some embodiments, positively charged moieties comprise amine groups, e.g., primary amine groups, secondary amine groups, tertiary amine groups, quaternary amine groups, or any combination thereof. Positively charged moieties may include any alkyl or aryl amine having a positive charge at a reference pH. Without wishing to be bound by theory, amine groups can be classified by the number of organic (e.g., alkyl or aryl) substituents the nitrogen atom is bonded with, e.g., a primary amine has a single organic substituent bonded to the nitrogen atom, a secondary amine has two organic substituents bonded to the nitrogen atom, and so forth. For example, a positively charged moiety comprising a quaternary amine may comprise a nitrogen atom attached to three methyl groups and having a fourth bond to the absorbent substrate (e.g., to a polysaccharide) or to a carbon that is connected to the absorbent substrate via one or more bonds (e.g., a series of bonds that connect to polysaccharide via a hydroxyl group of a sugar monomer). Such a quaternary amine group may also be referred to as a quaternary ammonium group, in reference to its positive charge; likewise, a tertiary amine group may also be referred to as a tertiary ammonium group in reference to its positive charge at reference pH.

In some embodiments, the amine groups comprise an alkylamine, arylamine, or alkylarylamine group. Exemplary amine groups include, but are not limited to: trimethylamino, dimethylamino, diethylaminoethyl (DEAE), diethylamino, dimethylamino, ethylmethylamino, propylethylamino, Tris (tris(hydroxymethyl)aminomethane), and ethylmethylaminomethyl groups. In some embodiments, the amine groups comprise a heterocyclic group comprising at least one nitrogen atom. Non-limiting examples of suitable heterocyclic groups include pyrrolidinyl, piperidinyl, and morpholino groups. In some embodiments, the heterocyclic group comprises an aromatic group (e.g., a heteroaromatic amine group). Non-limiting examples of suitable aromatic groups include pyrrolyl, imidazolyl, pyrazolyl, triazolyl, pyridinyl, and pyrimidinyl groups. Each amine group described herein may be substituted or unsubstituted. A person of skill in the art will recognize that numerous amines are known in the art and may be used as positively charged moieties in the methods and devices of the disclosure. In some embodiments, the positively charged moiety comprising an amine group comprises lysine.

In some embodiments, positively charged moieties comprise imines, e.g., ketimines or aldimines.

In some embodiments, positively charged moieties comprise metal ions, e.g., divalent metal ions or trivalent metal ions. Suitable metal ions include, but are not limited to, Ni²⁺, Zn²⁺, Cu²⁺, Ca²⁺, Co²⁺, Fe²⁺, Al³⁺, Ga³⁺, Fe³⁺, or Cr³⁺. In some embodiments, a metal ion is a lanthanide (e.g., lanthanum, cerium, praseodymium, neodymium, promethium, samarium, europium, gadolinium, terbium, dysprosium, holmium, erbium, thulium, ytterbium, or lutetium) ion. In some embodiments, a positively charged moiety comprises a metal ion and a chelating group. In some embodiments, the chelating group binds to the metal ion and is associated with the absorbent substrate (e.g., binds to the absorbent substrate or is covalently associated with the absorbent substrate). Exemplary chelating groups may comprise carboxylic acid groups (e.g., iminodiacetate ions). Examples of polysaccharides comprising chelating groups suitable for binding to biological polymers include immobilized metal chelate affinity chromatography media such as chelating Sepharose, or Chelex resin (e.g., chelex 100).

In some embodiments, a trap region comprising a plurality of positively charged moieties comprises a charged membrane. In some embodiments, the charged membrane binds to, e.g., selectively retains, a target (e.g., nucleic acid) in a fluid sample. In some embodiments, the charged membrane comprises a silica membrane or nylon membrane. In some embodiments, a charged membrane binds to a nucleic acid target in the fluid sample, e.g., to one, two, three, or all of labeled target amplification products, labeled control amplification products, labeled unextended primers, or unlabeled primers. In some embodiments, a charged membrane binds to a hapten or label of a labeled target amplification product, labeled control amplification product, and labeled unextended primer. In some embodiments, a charged membrane binds, e.g., non-specifically, to nucleic acid in a fluid sample.

### Absorbent Substrate

In some embodiments, the absorbent substrate comprises a polymer. In certain embodiments, the polymer is an acrylic or a styrenic polymer (e.g., polystyrene). In some instances, the acrylic polymer is a methyl acrylic polymer (e.g., poly(methyl acrylate), poly(methyl methacrylate)). In some embodiments, the polymer comprises a polysaccharide. In some embodiments, the polysaccharide comprises cellulose, nitrocellulose, dextrin, chitin, chitosan, or agarose (e.g., Sepharose). In some embodiments, the absorbent substrate comprises one or more free hydroxyl groups capable of reacting with a chemical reagent. In some embodiments, the absorbent substrate comprises an ion exchange resin, e.g., comprising a chelating group binding a positively charged ion. In some embodiments, the polymer comprises poly-lysine.

In some embodiments, the absorbent substrate comprises a first polymer and a second polymer, where one or both of the first and second polymers comprises a plurality of positively charged moieties. In some embodiments, the first and second polymers are associated non-covalently, e.g., physically associated but not via covalent chemical bonds. In some embodiments, the first and second polymers are associated covalently (e.g., cross-linked to each other). In some embodiments, the first polymer does not comprise a plurality of positively charged moieties and the second polymer comprises a plurality of positively charged moieties. In some embodiments, the second polymer comprises a plurality of amine groups. In some embodiments, the second polymer comprises polyethylenimine (PEI). In some embodiments, the second polymer comprises poly-lysine. For example, in some embodiments the absorbent substrate comprises a polysaccharide (e.g., cellulose) and a second polymer comprising a plurality of amine groups that are positively charged at a reference pH. In some embodiments, the absorbent substrate comprises cellulose and PEI. In some embodiments, the PEI comprises linear polyethylenimine. In some embodiments, the PEI comprises branched polyethylenimine. In some embodiments, the PEI is exposed to suitable conditions, e.g., alkaline conditions. In some embodiments, exposing PEI to alkaline conditions deacetylates monomers to expose amine groups. In some embodiments, the PEI is alkaline-treated PEI. In some embodiments, the absorbent substrate comprising a first polymer and a second polymer, where the second polymer comprises PEI, is exposed to alkaline conditions.

In some embodiments, an absorbent substrate comprising positively charged moieties is provided by contacting the absorbent substrate with a chemical reagent (e.g., in the presence of a base and/or chemical cross linker). In some embodiments, a method of preparing or making a lateral flow assay strip or a trap region comprised therein comprises contacting the sub-region comprising the trap region with a chemical reagent and/or chemical cross-linker. In some embodiments, the chemical cross-linker is chosen from a dihalohydrin (e.g., epichlorohydrin) or 1,4-butanediol diglycidyl ether. In some embodiments, the chemical reagent comprises glycidyltrimethylammonium chloride (GTMAC) or 2-chloro-N,N-diethylethanamine (see, e.g., R. Rousseau et al., Ind. Eng. Chem. Prob. Res. Dev., 1984, 23, 250-252, which is hereby incorporated by reference in its entirety). In some embodiments, the chemical reagent comprises diethylaminoethyl (DEAE). In some embodiments, a method of preparing or making a lateral flow assay strip or a trap region comprised therein comprises exposing the sub-region comprising the trap region to alkaline or acidic conditions, e.g., prior to or concurrently with contacting the sub-region with a chemical reagent and/or cross-linker. In some embodiments, contacting the sub-region comprising the trap region with a chemical reagent and/or cross-linker occurs under conditions suitable for transfer of a positively charged moiety to the absorbent substrate, e.g., alkaline or acidic conditions, e.g., the presence of a base or acid. In some embodiments, the suitable conditions (e.g., alkaline conditions) comprise a pH of 7-8, 7-9, 7-10, 7-11, 7-12, 7-13, 7-14, 8-9, 8-10, 8-11, 8-12, 8-13, 8-14, 9-10, 9-11, 9-12, 9-13, 9-14, 10-11, 10-12, 10-13, 10-14, 11-12, 11-13, 11-14, 12-13, 12-14, or 13-14. In some embodiments, the suitable conditions (e.g., alkaline conditions) comprise a pH of at least 8, 8.5, 9, 9.5, 10, 10.5, 11, 11.5, 12, 12.5, 13, 13.5, or 14, e.g., at least 10). For example, in some embodiments the chemical reagent comprises an epoxide and the suitable conditions comprise a pH of at least 10. In some embodiments, the suitable conditions (e.g., acidic conditions) comprise a pH of 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In some embodiments, an absorbent substrate or a polymer for use in an absorbent substrate is incubated in the presence of a chemical reagent and/or under suitable conditions for providing a positively charged moiety. In some embodiments, incubation occurs at room temperature. In some embodiments, incubation occurs at approximately 20, 25, 30, 35, 37, 40, 42, 45, 50, 55, 60, 65, 70, or 75° C. As used herein, approximately refers to being within a reference range of a specified value. In some embodiments, approximately refers to being within 1, 5, 10, 15, 20, or 25% of a specified value, e.g., within 5 or 10% of a specified value, e.g., 10%.

In some embodiments, wherein the absorbent substrate comprises one or more sugar monomers, the chemical reagent covalently attaches an ammonium group to one or more hydroxyl groups of the sugar monomers. For example, an exemplary absorbent substrate, cellulose, comprises sugar monomers comprising hydroxyl groups. Treatment of cellulose with chemical reagents comprising suitable nucleophilic targets after alkali-activation of the sugar hydroxyl groups may covalently attach the chemical reagent (e.g., a positively charged moiety, e.g., an ammonium group) to the cellulose. Examples of surface modifications to cellulose, e.g., to produce cationic cellulose, can be found in Courtenay et al. Cellulose (2017) 24:253-267; Courtenay et al. Cellulose (2018) 25:925-940; and Orlando et al. Front. Bioeng. Biotechnol., 24 Sep. 2020; Vol. 8:557885, each of which is incorporated by reference in its entirety. An example of the use of DEAE-modified cellulose can be found in Winberg and Hammarskjold. Nucleic Acids Res. 1980 Jan. 25; 8(2): 253-264, which is incorporated by reference in its entirety. An example of the use of dextran modified with a positively charged moiety can be found in U.S. Pat. No. 4,591,638, which is incorporated by reference in its entirety. Exemplary chemical reagents include suitable reactive groups (e.g., epoxide) operably linked to positively charged moieties or moieties expected to be charged upon reaction with the absorbent substrate and at a reference pH (e.g., neutral pH, the conditions of a method described herein, or the pH of a fluid sample or amplification product mixture). Exemplary chemical reagents include but are not limited to GTMAC and 2-chloro-N,N-diethylethanamine (also known as 2-chlorotriethylamine).

In some embodiments, the absorbent substrate comprises a polysaccharide comprising chitosan. Chitosan comprises β-(1→4)-linked D-glucosamine and N-acetyl-D-glucosamine. In some embodiments, chitosan is produced by treating chitin with a chemical reagent or suitable conditions (e.g., alkaline or acidic conditions). In some embodiments, chitosan is produced without the use of a chemical reagent, e.g., by treating chitin with suitable conditions (e.g., alkaline or acidic conditions). Chitin is a polymer of N-acetyl-D-glucosamine. In some embodiments, suitable conditions comprise alkaline conditions, e.g., treatment with NaOH. Without wishing to be bound by theory, the presence of a base deacetylates the N-acetyl-D-glucosamine monomers of chitin to produce chitosan which comprises β-(1→4)-linked D-glucosamine monomers comprising amine groups having a pKa of approximately 6.5, making them significantly positively charged at neutral and mildly acidic pH.

### Methods of Preparing Nucleic Acid Binding Moieties

In another aspect, the disclosure is directed, in part, to a method of preparing a lateral flow assay strip comprising a trap region. In some embodiments, the lateral flow assay strip comprising a trap region is a lateral flow assay strip described herein, e.g., comprising a first sub-region wherein the fluid sample can be introduced to the lateral flow assay strip, wherein the first sub-region comprises a trap region comprising a plurality of positively charged moieties capable of binding nucleic acid. In some embodiments, the method comprises contacting a sub-region of a lateral flow assay strip (e.g., the first sub-region or fourth sub-region) with a chemical reagent comprising a positively charged moiety under conditions suitable for transferring the positively charged moiety to a moiety of the absorbent substrate, thereby providing a trap region, e.g., in the first sub-region or fourth sub-region.

In another aspect, the disclosure is directed, in part, to a method of making a lateral flow assay strip comprising a trap region. In some embodiments, the lateral flow assay strip comprising a trap region is a lateral flow assay strip described herein, e.g., comprising a first sub-region wherein the fluid sample can be introduced to the lateral flow assay strip, wherein the first sub-region comprises a trap region comprising a plurality of positively charged moieties capable of binding nucleic acid. In some embodiments, the method comprises providing a lateral flow assay strip (e.g., comprising an absorbent substrate having a first end and a second end, wherein the substrate is configured to move a fluid sample from the first end to the second end by capillary action, and a plurality of sub-regions). In some embodiments, the method comprises contacting a sub-region of a lateral flow assay strip (e.g., the first sub-region or fourth sub-region) with a chemical reagent comprising a positively charged moiety under conditions suitable for transferring the positively charged moiety to a moiety of the absorbent substrate, thereby providing a trap region, e.g., in the first sub-region or fourth sub-region.

In some embodiments, providing comprises manufacturing the lateral flow assay strip. In some embodiments, providing comprises acquiring the lateral flow assay strip, e.g., from a third party.

In some embodiments, a chemical reagent comprises an epoxide. In some embodiments, the chemical reagent comprises an amine, e.g., a quaternary or tertiary amine. In some embodiments, the contacting step further comprises contacting the sub-region (e.g., the first or fourth sub-region) with a chemical cross-linker, e.g., dihalohydrin (e.g., epichlorohydrin) or 1,4-butanediol diglycidyl ether. In some embodiments, the chemical reagent comprises glycidyltrimethylammonium chloride (GTMAC). In some embodiments, the chemical reagent comprises 2-chloro-N,N-diethylethanamine (also known as 2-chlorotriethylamine). In some embodiments, the chemical reagent comprises diethylaminoethyl (DEAE). In some embodiments, the conditions suitable for transfer comprise alkaline conditions and/or the presence of a base (e.g., NaOH), e.g., conditions comprising a pH of 7-8, 7-9, 7-10, 7-11, 7-12, 7-13, 7-14, 8-9, 8-10, 8-11, 8-12, 8-13, 8-14, 9-10, 9-11, 9-12, 9-13, 9-14, 10-11, 10-12, 10-13, 10-14, 11-12, 11-13, 11-14, 12-13, 12-14, or 13-14. In some embodiments, the conditions suitable for transfer comprise acidic conditions and/or the presence of an acid (e.g., HCl), e.g., conditions comprising a pH of 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2.

In some embodiments, contacting a sub-region with a chemical reagent comprises incubating the sub-region with the chemical reagent. In some embodiments, incubation has a duration of at least 1, 2, 3, 4, 5, 10, 20, 30, 40, 50, or 60 minutes, or 1, 2, 3, 4, 5, 6, 8, 10, 12, 16, 20, or 24 hours (and optionally no more than 36, 24, 12, 10, 8, 6, 4, 2, or 1 hours, or 60, 40, 30, 20, 10, 5, 4, 3, or 2 minutes). In some embodiments, incubation occurs at room temperature.

In some embodiments, a method of making or preparing a lateral flow assay strip comprises a rinsing step comprising rinsing the sub-region until a neutral pH is achieved. In some embodiments, rinsing occurs after the contacting step. In some embodiments, rinsing comprises contacting or submerging the first sub-region in a neutral pH liquid, e.g., distilled water or a neutral pH buffer solution.

### Trap Reagents

In some embodiments, a fluid sample, e.g., that has undergone nucleic acid amplification, comprises labeled target amplification products, labeled control amplification products, and/or labeled unextended primers. In some embodiments, a labeled target amplification product comprises a target nucleic acid-specific primer (e.g., comprising a first hapten) extended by an amplification reaction. In some embodiments, a labeled control amplification product comprises a control nucleic acid-specific primer (e.g., comprising a second hapten) extended by an amplification reaction. In some embodiments, a labeled unextended primer comprises either a target nucleic acid-specific primer (e.g., comprising a first hapten) or a control nucleic acid-specific primer (e.g., comprising a second hapten) that have not undergone extension by an amplification reaction. In some embodiments, a detection method or composition (e.g., a lateral flow assay strip or device comprising the same) described herein detects one or more target nucleic acid sequences based upon detecting a labeled target amplification product. In some embodiments, detecting the one or more target nucleic acid sequences is further based upon detecting a labeled control amplification product.

As used herein, a trap reagent refers to an agent capable of specifically binding to a target (e.g., a hapten, an extension blocking moiety, label moiety, or nucleic acid) in a fluid sample. In some embodiments, the concentration of the target in the fluid sample flowing through a trap region of a lateral flow assay strip comprising one or more trap reagents is decreased. Without wishing to be bound by theory, a trap region comprising one or more trap reagents is thought to selectively bind to the target and decrease the level of the target in the fluid sample prior to the fluid sample encountering one or more test lines on a lateral flow assay strip.

In some embodiments, the trap reagent is immobilized, e.g., on a trap region of a lateral flow assay strip. In some embodiments, a trap reagent is not immobilized, e.g., is present in solution, e.g., in a trap solution. In some embodiments, a trap reagent specifically binds to a nucleic acid sequence present in the fluid sample, e.g., with specificity to a hapten component of the nucleic acid or with specificity to the sequence of the nucleic acid.

In some embodiments, the one or more trap reagents specifically bind one, two, three, or all of the labeled target amplification products, labeled control amplification products, labeled unextended primers, or unlabeled primers. In some embodiments, at least one of the one or more trap reagents specifically binds to a first hapten. In some embodiments, one, two, or all of the labeled target amplification products, labeled control amplification products, and labeled unextended primers comprise the first hapten. In some embodiments, the first hapten comprises a Fluorescein isothiocyanate (FITC) molecule. In some embodiments, at least one of the one or more trap reagents specifically binds to a second hapten. In some embodiments, one, two, or all of the labeled target amplification products, labeled control amplification products, and labeled unextended primers comprise the second hapten. In some embodiments, the second hapten comprises digoxigenin (DIG). In some embodiments, at least one of the one or more trap reagents specifically binds to a label moiety. In some embodiments, one, two, or all of the labeled target amplification products, labeled control amplification products, and labeled unextended primers comprise the label moiety. In some embodiments, the label moiety comprises biotin. In some embodiments, the particle conjugate pad comprises a plurality of labeled particles, wherein the labeled particles bind to the label moiety.

In some embodiments, the trap reagent comprises: an antibody, an antigen-binding fragment (e.g., a single-chain variable fragment (scFv)), an aptamer, or a functional portion of either thereof; an oligonucleotide complementary to a portion of one, two, three, or all of the labeled target amplification products, labeled control amplification products, labeled unextended primers, or unlabeled primers; a dendrimer comprising a plurality of oligonucleotides complementary to a portion of one, two, three, or all of the labeled target amplification products, labeled control amplification products, labeled unextended primers, or unlabeled primers; an enzyme (e.g., a phosphatase or nuclease, e.g., shrimp alkaline phosphatase or exonuclease I); a size exclusion resin; or an ion exchange resin.

In some embodiments, the annealing temperature of an oligonucleotide trap reagent to one, two, three, or all of the labeled target amplification products, labeled control amplification products, labeled unextended primers, or unlabeled primers is approximately room temperature.

In some embodiments, the target of a trap reagent or charged membrane is a labeled target amplification product. In some embodiments, the target of a trap reagent or charged membrane is a labeled control amplification product. In some embodiments, the target of a trap reagent or charged membrane is a labeled unextended primer. In some embodiments, the target of a trap reagent or charged membrane comprises two, or all of labeled target amplification products, labeled control amplification products, and labeled unextended primers.

In some embodiments, the target of a trap reagent is an extension blocking group (e.g., comprised on a target nucleic acid-specific primer or control nucleic acid-specific primer). Without wishing to be bound by theory, in an amplified fluid sample, an extension blocking group would only be expected to be present on labeled unextended primers if the amplification reaction employed a polymerase capable of removing the extension blocking group. A trap reagent that targets an extension blocking group would thus be expected to selectively target labeled unextended primers over labeled target amplification products or labeled control amplification products. In some embodiments, a trap reagent targets an extension blocking group and specifically binds to labeled unextended primers. In some embodiments, a trap reagent targets an extension blocking group and does not appreciably target labeled target amplification products or labeled control amplification products. In some embodiments, the extension blocking group is positioned at the 3′ end of the labeled unextended primers. In some embodiments, the extension blocking group is not present on the labeled target amplification products or labeled control amplification products. In some embodiments, the trap reagent that binds to the extension blocking group binds to labeled unextended primers but not to labeled target amplification products or labeled control amplification products. In some embodiments, the extension blocking group comprises a nucleotide from which polymerization cannot proceed. In some embodiments, the extension blocking group comprises a chemical moiety that blocks polymerization. In some embodiments, the extension blocking group comprises a dideoxynucleotide, a modified nucleotide, or a nucleotide analog. In some embodiments, the extension blocking group can be removed from a labeled unextended primer by an exonuclease.

### Methods of Detection

In another aspect, the disclosure is directed, in part, to a method for detecting a target nucleic acid in a fluid sample, comprising contacting a lateral flow assay strip described herein with a fluid sample, e.g., comprising an amplification product mixture comprising labeled target amplification products, under conditions under which a target nucleic acid or labeled target amplification product binds to the first test line to produce a first detectable signal. In some embodiments, contacting occurs under conditions under which a labeled target amplification product which may be present in the amplification product mixture can bind to the one or more test lines to produce a first detectable signal. In some embodiments, the method further comprises identifying the presence of the target nucleic acid in the fluid sample based upon detecting the presence of the first detectable signal, or identifying the absence of the target nucleic acid in the fluid sample based on detecting the absence of the first detectable signal.

In some embodiments, the fluid sample further comprises labeled control amplification products. In some embodiments, the labeled control amplification products bind to the second test line to produce a second detectable signal. In some embodiments, identifying the presence or absence of the target nucleic acid in the fluid sample is further based upon detecting the presence of the second detectable signal.

In some embodiments, a fluid sample comprises an amplification product mixture comprising labeled target amplification products. In some embodiments, a method described herein further comprises performing an isothermal amplification reaction on a biological sample (e.g., a biological sample comprising a target gene), thereby producing an amplification product mixture comprising labeled target amplification products. In some embodiments, the biological sample further comprises a control gene, e.g., from which is produced a labeled control amplification product.

In some embodiments, the amplification reaction is performed in a buffer solution comprising one or more biotinylated oligonucleotides, a target nucleic acid-specific primer comprising a first hapten, and a control nucleic acid-specific primer comprising a second hapten different from the first hapten. In some embodiments, the amplification product mixture further comprises labeled control amplification products. In some embodiments, contacting a lateral flow assay strip with a fluid sample occurs under conditions such that the labeled control amplification products bind to the second test line to produce a second detectable signal.

In some embodiments, the one or more biotinylated oligonucleotides, the target nucleic acid-specific primer, the control nucleic acid-specific primer, or a combination of any thereof comprise an extension blocking group which can be removed by an exonuclease. In some embodiments, the amplification reaction is performed by a polymerase comprising exonuclease activity capable of removing the extension blocking group. In some embodiments, the labeled target amplification products and labeled control amplification products do not comprise the extension blocking group. In some embodiments, the amplification product mixture comprises labeled unextended primers comprising the extension blocking group.

### Solution Phase Methods and Trap Solutions

The disclosure is also directed, in part, to a method for detecting target nucleic acids in a biological sample, comprising contacting an amplification product mixture with a trap solution comprising one or more trap reagents. In some embodiments, contacting the amplification product mixture with the trap solution forms a trap-treated amplification product mixture. In some embodiments, the trap-treated amplification product mixture comprises a lower level of one, two, three, or all of labeled target amplification products, labeled target control products, labeled unextended primers, or unlabeled primers than the amplification product mixture. In some embodiments, the trap-treated amplification product mixture comprises a lower level of free labeled target amplification products, labeled target control products, labeled unextended primers, or unlabeled primers, where free refers to the product or primer unbound by a trap reagent, than the amplification product mixture. Without wishing to be bound by theory, it is thought that contacting an amplification product mixture with a trap solution comprising one or more trap reagents can decrease the level of labeled product, unextended primer, or unlabeled primer in a manner similar to the effect flowing a fluid sample through a sub-region of a lateral flow assay strip comprising a trap region.

In some embodiments, a method that contacts an amplification product mixture with a trap solution comprises performing a method of amplifying a nucleic acid (e.g., an isothermal amplification reaction) on the biological sample to produce the amplification product mixture (e.g., comprising one, two, three, or all of labeled target amplification products, labeled target control products, labeled unextended primers, or unlabeled primers). In some embodiments, performing a method of amplifying a nucleic acid occurs prior to contacting an amplification product mixture with a trap solution.

In some embodiments, contacting the amplification product mixture with the trap solution comprises contacting the amplification product mixture with one or more trap reagents in solid form. In some embodiments, contacting the amplification product mixture with the trap solution comprises contacting the amplification product mixture with one or more trap reagents in aqueous form. In some embodiments, contacting comprises mixing the trap solution with the amplification product mixture, e.g., by pipetting or inversion of a reaction tube. In some embodiments, contacting the amplification product mixture with the trap solution comprises pipetting the amplification product mixture into a reaction tube comprising the trap solution. In some embodiments, contacting the amplification product mixture with the trap solution comprises pipetting the trap solution into a reaction tube comprising the amplification product mixture. In some embodiments, contacting the amplification product mixture with the trap solution comprises contacting the amplification product mixture with the one or more trap reagents in solid form (e.g., dissolving the one or more trap reagents into the amplification product mixture to form a trap-treated amplification product mixture). In some embodiments, the one or more trap reagents are present in the trap-treated amplification product mixture at a concentration less than the concentration of a labeled target amplification product or labeled control amplification product, e.g., the concentration of biotinylated or hapten-labeled oligonucleotide. In some embodiments, the one or more trap reagents are present in the trap-treated amplification product mixture at a concentration less than the concentration of the target nucleic acid-specific primer comprising a first hapten. In some embodiments, the one or more trap reagents are present in the trap-treated amplification product mixture at a concentration less than the concentration of the control nucleic acid-specific primer comprising a second hapten.

In some embodiments, a method described herein comprises contacting an amplification product mixture with a trap solution comprising one or more trap reagents to form a trap-treated amplification product mixture. Without wishing to be bound by theory, the majority (e.g., the overwhelming majority, nearly all, or all) of a decrease in the level of an amplification product mixture component (e.g., a labeled target amplification product, a labeled control amplification product, or a labeled unextended primer) achieved through such a contacting step is thought to be due to the one or more trap reagents. In some embodiments, such a contacting step decreases the level of an amplification product mixture component (e.g., a labeled target amplification product, a labeled control amplification product, or a labeled unextended primer) by dilution and use of the one or more trap reagents. In some embodiments, such a contacting step decreases the level of an amplification product mixture component (e.g., a labeled target amplification product, a labeled control amplification product, or a labeled unextended primer) by use of the one or more trap reagents. In some embodiments, such a contacting step does not decrease the level of an amplification product mixture component (e.g., a labeled target amplification product, a labeled control amplification product, or a labeled unextended primer) by dilution. A person of skill in the art will recognize that volumes and concentrations of reagents for use in methods or devices described herein can be adjusted to decrease, minimize, or eliminate dilution of sample or reaction components.

In some embodiments, the one or more trap reagents are present in the trap-treated amplification product mixture at a concentration less than the concentration of biotinylated oligonucleotide. In some embodiments, the one or more trap reagents are present in the trap-treated amplification product mixture at a concentration less than the concentration of the target nucleic acid-specific primer (e.g., comprising a first hapten). In some embodiments, the one or more trap reagents are present in the trap-treated amplification product mixture at a concentration less than the concentration of the control nucleic acid-specific primer (e.g., comprising a second hapten). Without wishing to be bound by theory, the concentration of the one or more trap reagents in a trap solution may be selected such that, upon contact with an amplification product mixture, the trap reagents decreases the level of one or both of the labeled target amplification products or the labeled control amplification products, without decreasing those levels to the extent that it impairs obtaining a detectable signal in a downstream immunoassay (e.g., on a lateral flow assay strip described herein). In other words, the concentration of trap reagent in a trap solution may be titrated to decrease the level of labeled products and/or labeled primer sufficiently to produce a more intense signal on an immunoassay, without decreasing the labeled products so much as to produce a weaker signal in said immunoassay.

In some embodiments, the one or more trap reagents of a trap solution comprises a size exclusion resin. In some embodiments, the size exclusion resin comprises a plurality of beads. In some embodiments, the size exclusion resin comprises polyacrylamide or agarose. In some embodiments, the size exclusion resin comprises G-50, Sephacryl S-200, or Sephacryl S-300. Without wishing to be bound by theory, labeled unextended primers are, in some cases, thought to be smaller than labeled target amplification products or labeled control amplification products, such that size exclusion resin may selectively retain labeled unextended primers while not retaining the labeled target amplification products or labeled control amplification products. In other cases, labeled target amplification products may be differently sized compared to labeled control amplification products, such that size exclusion resin may selectively retain one or the other. In some embodiments, the size exclusion resin comprises an average pore size configured to selectively retain labeled unextended primer relative to labeled target amplification products, labeled control amplification products, or both. In some embodiments, the size exclusion resin comprises an average pore size configured to selectively retain labeled target amplification products relative to labeled target unextended primer, labeled control amplification products, or both. In some embodiments, the size exclusion resin comprises an average pore size configured to selectively retain labeled control amplification products relative to labeled target unextended primer, labeled target amplification products, or both. In some embodiments, the size exclusion resin comprises an average pore size of at least 400, 500, 600, 700, 800, 900, or 1000 angstroms, e.g., at least 500 angstroms.

In some embodiments, the one or more trap reagents of a trap solution comprises a charged molecule, e.g., anion or cation exchange resin. In some embodiments, the one or more trap reagents comprise a positively charged molecule. In some embodiments, the one or more trap reagents comprise anion exchange resin, e.g., diethylaminoethyl resin. Without wishing to be bound by theory, nucleic acids such as labeled unextended primers, labeled target amplification products, or labeled control amplification products can be long, polymeric charged molecules which may be separated based on length/size using ion exchange chromatography resin. In some embodiments, a charged molecule (e.g., anion exchange resin) is used to selectively retain labeled unextended primer relative to labeled target amplification products, labeled control amplification products, or both. In some embodiments, a charged molecule (e.g., anion exchange resin) is used to selectively retain labeled target amplification products relative to labeled target unextended primer, labeled control amplification products, or both. In some embodiments, a charged molecule (e.g., anion exchange resin) is used to selectively retain labeled control amplification products relative to labeled target unextended primer, labeled target amplification products, or both.

In some embodiments, a method for detecting target nucleic acids in a biological sample comprises contacting an amplification product mixture with a trap solution comprising one or more trap reagents, and subsequently contacting a lateral flow assay strip comprising a sub-region comprising a trap region with the trap-treated amplification product mixture. In some embodiments, the trap solution comprises a first trap reagent and the trap region comprises a second trap reagent. In some embodiments, the first trap reagent binds to one, two, three, or all of the labeled unextended primers, labeled target amplification products, labeled control amplification products, or unlabeled primer (e.g., to a hapten, an extension blocking moiety, label moiety, or nucleic acid of the primer or product). In some embodiments, the second trap reagent binds to the first trap reagent. In some embodiments, the first trap reagent of the trap solution and the second trap reagent of the trap region function analogously to primary and secondary antibodies in an immunoassay. Without wishing to be bound by theory, it is thought that the first trap reagent of the trap solution may bind to one, two, three, or all of the labeled unextended primers, labeled target amplification products, labeled control amplification products, or unlabeled primer (e.g., to a hapten, an extension blocking moiety, label moiety, or nucleic acid), and then the second trap reagent of the trap region of a lateral flow assay strip can capture the labeled product or primer that has been tagged by the first trap reagent. Similar to other methods described herein, the result is thought to be a decrease in the level of one, two, three, or all of the labeled unextended primers, labeled target amplification products, labeled control amplification products, or unlabeled primer in the fluid sample and a more intense detectable signal.

### Exemplary Lateral Flow Assay Strip Embodiments

In some embodiments, the lateral flow assay strip comprises a first sub-region (e.g., a sample pad), a second sub-region (e.g., a particle conjugate pad) comprising a plurality of labeled particles, a third sub-region (e.g., a test pad) comprising one or more test lines, and a fourth sub-region comprising a trap region (e.g., comprising a trap pad or one or more trap lines).

In some embodiments, the fluid sample is introduced to the first sub-region (e.g., a sample pad) of the lateral flow assay strip. In some embodiments, the fluid sample subsequently flows through the second sub-region (e.g., a particle conjugate pad) comprising a plurality of labeled particles. In some cases, as an amplicon-containing fluid sample flows through the second sub-region (e.g., a particle conjugate pad), a labeled nanoparticle binds to a label of an amplicon, thereby forming a particle-amplicon conjugate. In some embodiments, the fluid sample subsequently flows through the fourth sub-region comprising a trap region (e.g., comprising a trap pad or one or more trap lines). In some cases, as a fluid sample comprising labeled target amplification products, labeled control amplification products, or labeled unextended primers flows through the fourth sub-region (e.g., trap pad or one or more trap lines), a trap reagent binds one or more of the labeled target amplification products, labeled control amplification products, or labeled unextended primers, thereby decreasing the level of labeled target amplification products, labeled control amplification products, or labeled unextended primers in the fluid sample. In some embodiments, the fluid sample subsequently flows through the third sub-region (e.g., a test pad) comprising one or more test lines. Accordingly, in some embodiments, the fluid sample is introduced to the first sub-region and flows, in order, to the second sub-region, the fourth sub-region, and the third sub-region.

In other embodiments, the fluid sample is introduced to the first sub-region (e.g., a sample pad) of the lateral flow assay strip. In some embodiments, the fluid sample subsequently flows through the fourth sub-region comprising a trap region (e.g., comprising a trap pad or one or more trap lines). In some cases, as a fluid sample comprising labeled target amplification products, labeled control amplification products, or labeled unextended primers flows through the fourth sub-region (e.g., trap pad or one or more trap lines), a trap reagent binds one or more of the labeled target amplification products, labeled control amplification products, or labeled unextended primers, thereby decreasing the level of labeled target amplification products, labeled control amplification products, or labeled unextended primers in the fluid sample. In some embodiments, the fluid sample subsequently flows through the second sub-region (e.g., a particle conjugate pad) comprising a plurality of labeled particles. In some cases, as an amplicon-containing fluid sample flows through the second sub-region (e.g., a particle conjugate pad), a labeled nanoparticle binds to a label of an amplicon, thereby forming a particle-amplicon conjugate. In some embodiments, the fluid sample subsequently flows through the third sub-region (e.g., a test pad) comprising one or more test lines. Accordingly, in some embodiments, the fluid sample is introduced to the first sub-region and flows, in order, to the fourth sub-region, the second sub-region, and the third sub-region.

In some embodiments, the lateral flow assay strip does not comprise a first sub-region (e.g., a sample pad). In such embodiments, the fluid sample may be introduced to the second sub-region (e.g., a particle conjugate pad) comprising a plurality of labeled particles or to the fourth sub-region comprising a trap region (e.g., comprising a trap pad or one or more trap lines).

In certain embodiments, the lateral flow assay strip comprises a fifth sub-region (e.g., a wicking area) to absorb fluid flowing through the lateral flow assay strip. Any excess fluid may flow through the fifth sub-region. In some embodiments, the fifth sub-region is the last sub-region the fluid sample flows through. In some embodiments, the fluid sample flows to the fifth sub-region after the fluid sample flows through the third sub-region (e.g., the test pad).

As an illustrative example, a fluid sample comprising an amplicon labeled with biotin and FITC may be introduced into a lateral flow assay strip (e.g., through a sample pad of a lateral flow assay strip). In some embodiments, as the labeled amplicon is transported through the lateral flow assay strip (e.g., through a trap region) one or more trap reagents bind to the biotin or the FITC, decreasing the level of labeled amplicon and/or labeled unextended primers in the fluid sample. In some embodiments, as the labeled amplicon is transported through the lateral flow assay strip (e.g., through a particle conjugate pad of the lateral flow assay strip), a gold nanoparticle labeled with streptavidin may bind to the biotin label of the amplicon. In some cases, the lateral flow assay strip (e.g., a test pad of the lateral flow assay strip) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay strip (e.g., a test pad of the lateral flow assay strip) further comprises a first lateral flow control line comprising biotin. In some embodiments, excess gold nanoparticles labeled with streptavidin (i.e., gold nanoparticles that were not conjugated to an amplicon) transported through the lateral flow assay strip may bind to the biotin of the first lateral flow control line, demonstrating that liquid was successfully transported to the first lateral flow control line. In some embodiments, one or more target nucleic acid sequences are detected using a colorimetric assay. In certain embodiments, for example, a fluid sample is exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluid sample is mixed with the reagents, the solution turns a specific color (e.g., red) if the target nucleic acid is present, and the sample is positive. If the solution turns a different color (e.g., green), the target nucleic acid is not present, and the sample is negative. In some embodiments, the colorimetric assay may be a colorimetric LAMP assay; that is, the LAMP reagents may react in the presence or absence of a target nucleic acid sequence (e.g., from SARS-CoV-2) to turn one of two colors.

In some embodiments, one, two, or all of the first sub-region, the second sub-region, or fourth sub-region comprise one or more buffer components in solid form configured to dissolve into the fluid sample. In some embodiments, the fourth sub-region comprises one or more buffer components in solid form configured to dissolve into the fluid sample. In some embodiments, the first sub-region, second sub-region, or both comprise one or more buffer components in solid form configured to dissolve into the fluid sample. In some embodiments, a lateral flow assay strip does not comprise a fourth sub-region comprising a trap region, wherein the first sub-region, the second sub-region, or both comprise one or more buffer components in solid form configured to dissolve into the fluid sample. In some embodiments, the one or more buffer components in solid form are lyophilized, dried, crystallized, or air jetted. The disclosure is directed, in part, to the discovery that the pH of a fluid sample may change as the fluid sample flows from sub-region to sub-region of a lateral flow assay strip. Without wishing to be bound by theory, it is thought that an approximately neutral or slightly basic pH in the fluid sample promotes immunocomplex (a complex comprising an antibody and the epitope to which it binds) formation on the lateral flow assay strip (e.g., in the third sub-region comprising one or more test lines). The disclosure is directed, in part, to compositions and methods using a lateral flow assay strip where one or more buffer components in solid form configured to dissolve into a fluid sample are disposed in one or more sub-regions of the strip such that the pH of a fluid sample is adjusted as it flows through the one or more sub-regions (e.g., to increase or decrease the pH to a neutral or slightly basic pH).

In some embodiments, the one or more buffer components in solid form is disposed in the first sub-region (e.g., comprising a sample pad where the fluid sample can be introduced to the lateral flow assay strip). In some embodiments, the one or more buffer components in solid form is disposed in the second sub-region (e.g., comprising a particle conjugate pad). In some embodiments, the one or more buffer components in solid form is disposed in the first sub-region (e.g., comprising a sample pad where the fluid sample can be introduced to the lateral flow assay strip) and the second sub-region (e.g., comprising a particle conjugate pad). In some embodiments, the one or more buffer components in solid form is disposed in the fourth sub-region (e.g., comprising a trap region). In some embodiments, the one or more buffer components in solid form is disposed in the first sub-region (e.g., comprising a sample pad where the fluid sample can be introduced to the lateral flow assay strip) and the fourth sub-region (e.g., comprising a trap region). In some embodiments, the one or more buffer components in solid form is disposed in the fourth sub-region (e.g., comprising a trap region) and the second sub-region (e.g., comprising a particle conjugate pad). In some embodiments, the one or more buffer components in solid form is disposed in the first sub-region (e.g., comprising a sample pad where the fluid sample can be introduced to the lateral flow assay strip), the fourth sub-region (e.g., comprising a trap region), and the second sub-region (e.g., comprising a particle conjugate pad).

In some embodiments, the one or more buffer components are configured to adjust the pH of the fluid sample to 5-8, 5-7, 5-6, 6-8, 6-7, or 7-8. In some embodiments, the one or more buffer components are configured to adjust the pH of the fluid sample to 7-8. In some embodiments, the one or more buffer components are configured to increase the pH of the fluid sample by at least 0.25, 0.5, 0.75, 1, 1.5, or 2 pH units. In some embodiments, the one or more buffer components are configured to decrease the pH of the fluid sample by at least 0.25, 0.5, 0.75, 1, 1.5, or 2 pH units.

Any salt or buffer component known to those of skill in the art and suitable to adjust the pH of a fluid sample as described herein may be used in the compositions and methods described herein. In some embodiments, the one or more buffer components comprises a salt, e.g., magnesium acetate tetrahydrate, potassium acetate, and/or potassium chloride. In some embodiments, the one or more buffer components comprises phosphate buffered saline (PBS) or Tris. In some embodiments, the one or more buffer components may comprise a buffer or salt that is also a component of an amplification product mixture (e.g., an amplification product mixture used to amplify a sample prior to contacting a lateral flow assay strip with said amplified sample).

### Diagnostic Systems

The disclosure is directed, in part, to devices (e.g., lateral flow assay strips) and methods of detecting a target nucleic acid (e.g., using said devices) that obtain improved detection signals over previously used devices and methods. In some embodiments, the first detectable signal, second detectable signal, or both are at least 10, 20, 30, 40, 50, 60, 70, 80, 90, 100, 200, or 300% more intense than the signal that would be obtained in an otherwise similar method wherein the lateral flow assay strip does not comprise a trap region comprising one or more trap reagents. In some embodiments, the first detectable signal, second detectable signal, or both are at least 10, 20, 30, 40, 50, 60, 70, 80, 90, 100, 200, or 300% more intense than the signal that would be obtained in an otherwise similar method that does not contact the amplification product mixture with a trap solution comprising one or more trap reagents. In some embodiments, the device (e.g., a lateral flow assay strip or device comprising the same) or the method (e.g., using a device described herein) produces at least 10, 20, 30, 40, 50, 60, 70, 80, 90, or 100% fewer false negative results than an otherwise similar device or method wherein the lateral flow assay strip does not comprise a trap region comprising one or more trap reagents. In some embodiments, the the device (e.g., a lateral flow assay strip or device comprising the same) or the method (e.g., using a device described herein) produces at least 10, 20, 30, 40, 50, 60, 70, 80, 90, or 100% fewer false negative results than an otherwise similar device or method that does not contact the amplification product mixture with a trap solution comprising one or more trap reagents. In some embodiments, when the lateral flow assay strip is contacted with a fluid sample comprising a target nucleic acid, the lateral flow assay strip produces at least 10, 20, 30, 40, 50, 60, 70, 80, 90, or 100% fewer false negative results than an otherwise similar lateral flow assay strip not comprising a trap region comprising one or more trap reagents.

In another aspect, the disclosure is directed, in part, to a rapid test device that performs testing for at least one nucleic acid in a single test procedure, the device comprising a lateral flow assay strip described herein.

In some embodiments, the rapid test device comprises a housing (e.g., comprising the lateral flow assay strip). In some embodiments, the rapid test device comprises an amplification chamber accommodated in the housing and configured to receive a biological sample and to amplify the test sample using at least one amplification reagent. In some embodiments, the amplification chamber is in fluidic communication with the lateral flow assay strip.

In some embodiments, the rapid test device comprises a sealable test tube holding a fluid therein, the test tube being configured to receive a biological sample and one or more reactants to form a reactant mixture containing the test sample. In some embodiments, the rapid test device comprises a housing (e.g., comprising the lateral flow assay strip). In some embodiments, the housing is configured to obtain the reactant mixture via an input port. In some embodiments, the input port is in fluidic communication with the lateral flow assay strip.

In another aspect, the disclosure is directed, in part, to a kit comprising an immunoassay device, e.g., a rapid test device or a lateral flow assay strip, described herein. In some embodiments, the kit comprises instructions for using the lateral flow assay strip or rapid test device to test for a target nucleic acid in a biological sample. In some embodiments, the instructions comprise the steps of a method described herein.

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals.

Diagnostic devices, systems, and methods described herein are also highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers (trained or untrained), in homes, schools, medical offices, or businesses.

In some embodiments, the disclosure relates to rapid, self-administrable tests for detecting the presence of one or more target nucleic acids derived from one or more pathogens. A “self-administrable” test refers to a test in which all testing steps are performed by the subject of the test. In some embodiments, a “rapid test” refers to a test in which all testing steps (e.g., sample collection, lysis, isothermal amplification, detection, etc.) may be completed in less than 3 hours, less than 2 hours, or less than 1 hour.

In some embodiments, diagnostic devices described herein are relatively small. In certain cases, for example, a diagnostic device is approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

In some embodiments, any reagents contained within a diagnostic device or system described herein may be thermostabilized, and the diagnostic device or system may be shelf stable for a relatively long period of time.

The present disclosure provides diagnostic devices, systems, and methods for rapidly and in a home environment detecting one or more target nucleic acid sequences (e.g., a nucleic acid sequence of a pathogen, such as SARS-CoV-2 or an influenza virus). Such diagnostic devices, systems, and methods are also referred to herein as rapid test devices, systems, or methods, respectively. A diagnostic system, as described herein, may be self-administrable and comprise a sample-collecting component (e.g., a swab) and a diagnostic device. The diagnostic device may comprise a cartridge, a blister pack, and/or a “chimney” detection device, according to some embodiments. In some cases, the diagnostic device comprises a detection component (e.g., a lateral flow assay strip, a colorimetric assay), results of which are self-readable, or automatically read by a computer algorithm. In certain embodiments, the diagnostic device further comprises one or more reagents (e.g., lysis reagents, nucleic acid amplification reagents, CRISPR/Cas detection reagents). In certain other embodiments, the diagnostic system separately includes one or more reaction tubes comprising the one or more reagents. The diagnostic device may also comprise an integrated heater, or the diagnostic system may comprise a separate heater. The isothermal amplification technique employed yields not only fast but very accurate results.

In some embodiments, a diagnostic device comprises a housing comprising a detection component comprising a “chimney.” In certain embodiments, the “chimney” detection component comprises a chimney configured to receive a reaction tube. In certain embodiments, the “chimney” detection component comprises a puncturing component configured to puncture the reaction tube. The puncturing component may comprise one or more blades, needles, or other elements capable of puncturing a reaction tube. In certain embodiments, the “chimney” detection component comprises a lateral flow assay strip. As described herein, the lateral flow assay strip may comprise one or more test lines configured to detect one or more target nucleic acid sequences. In some embodiments, the lateral flow assay strip further comprises one or more control lines.

In some embodiments, the “chimney” detection component comprises a chimney, a front panel, and a bottom panel comprising a lateral flow assay strip and a puncturing component. The chimney and the front panel may be integrally formed or may be separately formed. The chimney, the front panel, and the back panel may be formed from any suitable material(s). In some cases, for example, the chimney, the front panel, and/or the back panel comprise one or more thermoplastic materials and/or metals. In some embodiments, the chimney, the front panel, and/or the back panel may be manufactured by injection molding, an additive manufacturing technique (e.g., 3D printing), and/or a subtractive manufacturing technique (e.g., laser cutting).

The chimney may have suitable dimensions to receive a reaction tube. In certain embodiments, the chimney has a height of 60 mm or less, 55 mm or less, 50 mm or less, 45 mm or less, 40 mm or less, 35 mm or less, 30 mm or less, 25 mm or less, 20 mm or less, 15 mm or less, or 10 mm or less. In some embodiments, the chimney has a height in a range from 10 mm to 20 mm, 10 mm to 30 mm, 10 mm to 40 mm, 10 mm to 50 mm, 10 mm to 60 mm, 20 mm to 30 mm, 20 mm to 40 mm, 20 mm to 50 mm, 20 mm to 60 mm, 30 mm to 40 mm, 30 mm to 50 mm, 30 mm to 60 mm, 40 mm to 50 mm, 40 mm to 60 mm, or 50 mm to 60 mm. In certain embodiments, the chimney has an inner diameter of 30 mm or less, 25 mm or less, 20 mm or less, 15 mm or less, 10 mm or less, or 5 mm or less. In some embodiments, the chimney has an inner diameter of 5 mm to 10 mm, 5 mm to 15 mm, 5 mm to 20 mm, 5 mm to 25 mm, 5 mm to 30 mm, 10 mm to 15 mm, 10 mm to 20 mm, 10 mm to 25 mm, 10 mm to 30 mm, 15 mm to 20 mm, 15 mm to 25 mm, 15 mm to 30 mm, or 20 mm to 30 mm.

In some embodiments, a diagnostic system comprises a sample-collecting component (e.g., a swab), a reaction tube comprising one or more reagents, and a “chimney” detection component. In some embodiments, the diagnostic system further comprises a heater, as described herein. In some embodiments, a reaction tube may be inserted into a “chimney” detection component following heating.

In some embodiments, the diagnostic system comprises one or more reagents (e.g., lysis reagents, nucleic acid amplification reagents, CRISPR/Cas detection reagents). In some instances, at least one reagent is contained within a diagnostic device (e.g., a cartridge, a blister pack, a “chimney” detection component) of a diagnostic system. In some instances, at least one reagent is provided separately from the diagnostic device. In certain cases, for example, the diagnostic system comprises one or more reaction tubes comprising the at least one reagent.

In certain embodiments, the one or more reagents comprise one or more lysis reagents. A lysis reagent generally refers to a reagent that promotes cell lysis either alone or in combination with one or more reagents and/or conditions (e.g., heating). In some embodiments, the one or more lysis reagents comprise an RNase inhibitor (e.g., a murine RNase inhibitor). In certain embodiments, the one or more reagents comprise one or more reverse transcription reagents. In some embodiments, the one or more reagents comprise one or more nucleic acid amplification reagents. In certain embodiments, the one or more nucleic acid amplification reagents comprise LAMP reagents, RPA reagents, or NEAR reagents.

In some embodiments, the one or more reagents comprise one or more buffers. Non-limiting examples of suitable buffers include phosphate-buffered saline (PBS) and Tris. In some embodiments, the one or more buffers have a relatively neutral pH. In some embodiments, the one or more buffers have a pH in a range from 5.0 to 6.0, 5.0 to 7.0, 5.0 to 8.0, 5.0 to 9.0, 6.0 to 7.0, 6.0 to 8.0, 6.0 to 9.0, 7.0 to 8.0, 7.0 to 9.0, or 8.0 to 9.0. In some embodiments, the one or more reagents comprise one or more salts. Non-limiting examples of suitable salts include magnesium acetate tetrahydrate, potassium acetate, and potassium chloride.

In some embodiments, at least one reagent is not contained within a diagnostic device, and a diagnostic system comprises one or more reaction tubes. The one or more reaction tubes may contain any reagent(s) described above. In some embodiments, the one or more reaction tubes comprise at least one reagent in liquid form. In some embodiments, the one or more reaction tubes comprise at least one reagent in solid form.

The reaction tubes, in some embodiments, further comprise at least one cap. In some embodiments, the reaction tube comprises a partially removable cap (e.g., a hinged cap) or one or more wholly removable caps (e.g., one or more screw-top caps, one or more stoppers). In some embodiments, the one or more caps comprise reagents in solid form (e.g., lyophilized, dried, crystallized, air jetted reagents).

In some embodiments, the fluidic contents of the reaction tube comprise a reaction buffer. In certain instances, the reaction buffer comprises one or more buffers. In a particular, non-limiting embodiment, the reaction buffer comprises 25 mM Tris buffer, 5% (w/v) poly(ethylene glycol) 35,000 kDa, 14 mM magnesium acetate tetrahydrate, 100 mM potassium acetate, and greater than 85% volume nuclease free water.

In certain embodiments, the diagnostic device (e.g., cartridge, blister pack, “chimney” detection component) comprises a detection component. In some embodiments, the detection component comprises a lateral flow assay strip or a colorimetric assay. In some embodiments, results of the lateral flow assay strip and/or colorimetric assay are read and/or analyzed by software (e.g., a mobile application).

In some embodiments, the diagnostic device comprises a lateral flow assay strip. In some embodiments, the lateral flow assay strip is configured to detect one or more target nucleic acid sequences. In certain cases, the lateral flow assay strip comprises one or more fluid-transporting layers comprising one or more materials that allow fluid transport (e.g., via capillary action). Non-limiting examples of suitable materials include polyethersulfone, cellulose, polycarbonate, nitrocellulose, sintered polyethylene, and glass fibers. In some embodiments, the one or more fluid-transporting layers of the lateral flow assay strip are of the same material as the absorbent substrate of the trap region. In some embodiments, the one or more fluid-transporting layers of the lateral flow assay strip are of different material than the absorbent substrate, e.g., a separate component from the absorbent substrate. In either embodiment, the trap region and the one or more fluid-transporting layers of the lateral flow assay strip may be in fluidic communication with one another.

In certain embodiments, the lateral flow assay strip comprises one or more sub-regions. In some instances, the lateral flow assay strip comprises a first sub-region (e.g., a sample pad) where a fluid sample is introduced to the lateral flow assay strip. In some instances, the lateral flow assay strip comprises a second sub-region (e.g., a particle conjugate pad) comprising a plurality of labeled particles.

In certain embodiments, the lateral flow assay strip comprises a third sub-region (e.g., a test pad) comprising one or more test lines. In some embodiments, a first test line comprises a capture reagent (e.g., an immobilized antibody) configured to detect a first target nucleic acid sequence. In certain embodiments, the lateral flow assay strip comprises one or more additional test lines. In some instances, each test line of the lateral flow assay strip is configured to detect a different target nucleic acid. In some instances, two or more test lines of the lateral flow assay strip are configured to detect the same target nucleic sequence.

In certain embodiments, the third sub-region (e.g., the test pad) of the lateral flow assay strip comprises one or more control lines.

In certain embodiments, the lateral flow assay strip comprises a fifth sub-region (e.g., a wicking area) to absorb fluid flowing through the lateral flow assay strip.

In some embodiments, the diagnostic device comprises a colorimetric assay. In certain embodiments, the colorimetric assay comprises a cartridge comprising a central sample chamber in fluidic communication with a plurality of peripheral chambers (e.g., at least four peripheral chambers). In some embodiments, each peripheral chamber comprises isothermal nucleic acid amplification reagents comprising a unique set of primers (e.g., primers specific for one or more target nucleic acid sequences, primers specific for a positive test control, primers specific for a negative test control).

In operation, a sample may be deposited in the central sample chamber. In some cases, the sample may be combined with a reaction buffer in the central sample chamber. In certain cases, the central sample chamber may be heated to lyse cells within the sample. In some cases, the lysate may be directed to flow from the central sample chamber to the plurality of peripheral chambers comprising unique primers. In some cases, a colorimetric reaction may occur in each peripheral chamber, resulting in varying colors in the peripheral chambers. In some cases, the results within each peripheral chamber may be visible (e.g., through a clear film or other covering).

The diagnostic system, in some embodiments, comprises a heater. In certain embodiments, the heater is integrated with the diagnostic device. In some embodiments, the diagnostic system comprises a separate heater (i.e., a heater that is not integrated with other system components). In some embodiments, the heater is configured to receive a reaction tube.

In some embodiments, the heater is pre-programmed with one or more protocols. In some embodiments, for example, the heater is pre-programmed with a lysis heating protocol and/or an amplification heating protocol. A lysis heating protocol generally refers to a set of one or more temperatures and one or more time periods that facilitate lysis of the sample. An amplification heating protocol generally refers to a set of one or more temperatures and one or more time periods that facilitate nucleic acid amplification. In some embodiments, the heater comprises an auto-start mechanism that corresponds to the temperature profile needed for lysis and/or amplification. That is, a user may insert a reaction tube into the heater, and the heater may automatically run a lysis and/or amplification heating protocol. In some embodiments, the heater is controlled by a mobile application.

In some embodiments, a diagnostic system comprises instructions associated with system components.

In some embodiments, the device may be a disposable, single-use device. In some cases, the device may further comprise at least one container in which the device is stored before being used in a test procedure. In some cases, the at least one container may be sealed to prevent contamination of the device.

In some embodiments, the device, e.g., the housing, may be comprised of a window through which the lateral flow assay strip is visible.

In some embodiments, one or more components of a diagnostic system comprise a unique label. In some cases, this may advantageously allow multiple samples to be run in parallel. For example, one or more components of the diagnostic system (e.g., reaction tube cap, detection component) may be labeled with the same label. In some embodiments, a copy of the label is given to a tested subject, so that the subject may later receive the results using the unique label. In this way, multiple tests (one for each unique subject) may be run in parallel without mixing up the samples.

### Instructions & Software

In some embodiments, a diagnostic system comprises instructions for using a diagnostic device and/or otherwise performing a diagnostic test method. The instructions may include instructions for the use, assembly, and/or storage of the diagnostic device and any other components associated with the diagnostic system. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications).

In some embodiments, the instructions are provided as part of a software-based application. In certain cases, the application can be downloaded to a smartphone or device, and then guides a user through steps to use the diagnostic device. In some embodiments, the instructions instruct a user when to add certain reagents and how to do so.

In some embodiments, a software-based application may be connected (e.g., via a wired or wireless connection) to one or more components of a diagnostic system.

In some embodiments, a diagnostic systems comprises or is associated with software to read, transmit, communicate, and/or analyze test results. In some embodiments, a device (e.g., a camera, a smartphone) is used to generate an image of a test result (e.g., one or more lines detectable on a lateral flow assay strip).

### Enumerated Embodiments

1. A lateral flow assay strip usable to identify at least one target nucleic acid, the lateral flow assay strip comprising:

an absorbent substrate having a first end and a second end, wherein the substrate is configured to move a fluid sample from the first end to the second end by capillary action, and

a plurality of sub-regions comprising:
a first sub-region wherein the fluid sample can be introduced to the lateral flow assay strip, wherein the first sub-region comprises a trap region comprising a plurality of positively charged moieties,
optionally a second sub-region comprising a particle conjugate pad, and
a third sub-region comprising one or more test lines, and

wherein the one or more test lines are configured to indicate the presence of at least one target nucleic acid by interaction with the fluid sample.

2. A method for detecting a target nucleic acid in a fluid sample, the method comprising:

(i) contacting a lateral flow assay strip with a fluid sample, wherein the lateral flow assay strip comprises:

an absorbent substrate having a first end and a second end, wherein the substrate is configured to move a fluid sample from the first end to the second end by capillary action, and

a plurality of sub-regions comprising:

a first sub-region wherein the fluid sample can be introduced to the lateral flow assay strip, wherein the first sub-region comprises a trap region comprising a plurality of positively charged moieties capable of binding nucleic acid,

optionally a second sub-region comprising a particle conjugate pad, and

a third sub-region comprising one or more test lines, and

wherein the contacting occurs under conditions under which a target nucleic acid or labeled target amplification product which may be present in the fluid sample can bind to the one or more test lines to produce a first detectable signal, and

(ii) identifying the presence of the target nucleic acid in the fluid sample based upon detecting the presence of the first detectable signal, or identifying the absence of the target nucleic acid in the fluid sample based on detecting the absence of the first detectable signal.

3. The lateral flow assay strip or method of either of paragraphs 1 or 2, wherein the plurality of positively charged moieties comprise amino groups.
4. The lateral flow assay strip or method of paragraph 3, wherein the amino groups comprise primary amine groups, secondary amine groups, tertiary amine groups, quaternary amine groups, or a combination of any thereof.
5. The lateral flow assay strip or method of any of paragraphs 1-3, wherein the plurality of positively charged moieties comprise one, two, three, four, or all of:

an amino group,

a heterocyclic group comprising a piperidinyl or morpholino group,

an aromatic group comprising an imidazolyl or pyridinyl group,

an imine group, or

a metal ion with a chelating group.

6. The lateral flow assay strip or method of paragraph 5, wherein the metal ions comprise:

divalent cations selected from the group consisting of Ni²⁺, Zn²⁺, Cu²⁺, Ca²⁺, Co²⁺, and Fe²⁺;

trivalent cations selected from the group consisting of Al³⁺, Ga³⁺, Fe³⁺, and Cr³⁺, or

a lanthanide ion.

7. The lateral flow assay strip or method of any of paragraphs 1-6, wherein the plurality of positively charged moieties comprise trimethylamino, dimethylamino, and/or diethylaminoethyl (DEAE) groups.
8. A method of preparing a lateral flow assay strip comprising a trap region,

wherein the lateral flow assay strip comprises:

an absorbent substrate having a first end and a second end, wherein the substrate is configured to move a fluid sample from the first end to the second end by capillary action, and a plurality of sub-regions comprising a first sub-region wherein the fluid sample can be introduced to the lateral flow assay strip;

the method comprising:

contacting the first sub-region with a chemical reagent comprising a positively charged moiety and/or exposing the first-sub region to suitable conditions for providing a positively charged moiety to the absorbent substrate, thereby providing a trap region in the first sub-region,

thereby preparing a lateral flow assay strip comprising a trap region.

9. The lateral flow assay strip or method of any of paragraphs 1-8, wherein the absorbent substrate comprises a first polymer comprising a polysaccharide, a methyl acrylic polymer, or a styrenic polymer.
10. The lateral flow assay strip or method of any of paragraphs 1-8, wherein the absorbent substrate comprises a first polymer comprising poly-lysine.
11. The lateral flow assay strip or method of paragraph 9, wherein the polysaccharide is selected from the group consisting of cellulose, dextrin, chitosan, chitin, agarose, and nitrocellulose.
12. The lateral flow assay strip or method of any of paragraphs 9-11, wherein the polysaccharide comprises a plurality of sugar monomers, and wherein a plurality of the sugar monomers are chemically modified to comprise the plurality of positively charged moieties.
13. The lateral flow assay strip or method of any of paragraphs 1-7 or 9-12, wherein the positively charged moieties were attached by contacting the first sub-region with a chemical reagent and/or exposing the absorbent substrate of the first sub-region to suitable conditions.
14. The lateral flow assay strip or method of any of paragraphs 8-13, wherein the suitable conditions comprise alkaline conditions.
15. The lateral flow assay strip or method of any of paragraphs 8-13, wherein the suitable conditions comprise acidic conditions.
16. The lateral flow assay strip or method of any of paragraphs 8-15, wherein the chemical reagent is selected from the group consisting of glycidyltrimethylammonium chloride (GTMAC) 2-chlorotriethylamine, and lysine.
17. The method of any of paragraphs 8-16, wherein the contacting step further comprises contacting the first sub-region with a chemical cross-linker selected from the group consisting of a dihalohydrin and 1,4-butanediol diglycidyl ether.
18. The lateral flow assay strip or method of any of paragraphs 1-17, wherein the first sub-region and/or trap region comprises a second polymer comprising polyethylenimine (PEI), poly-lysine, chitin, and/or chitosan.
19. The lateral flow assay strip or method of any of paragraphs 1-18, wherein the first sub-region is thicker than at least one adjacent sub-region.
20. The lateral flow assay strip or method of any of paragraphs 1-19, wherein the first sub-region and/or trap region comprises a trap pad comprising an absorbent substrate that is separate from the absorbent substrate of at least one adjacent sub-region while remaining in fluidic communication with at least one adjacent sub-region.
21. The method of any of paragraphs 2-7 or 9-20, further comprising:

performing an isothermal amplification reaction on the biological sample, wherein the biological sample comprises a target nucleic acid,

thereby producing an amplification product mixture comprising one, two, three, or all of the labeled target amplification products, labeled control amplification products, labeled unextended primers, or unlabeled primers.

22. The method of any of paragraphs 2-7 or 9-21, wherein:

the first detectable signal, second detectable signal, or both are at least 10, 20, 30, 40, 50, 60, 70, 80, 90, 100, 200, or 300% more intense than the signal that would be obtained in an otherwise similar method wherein the lateral flow assay strip does not comprise a trap region;

the method produces at least 10, 20, 30, 40, 50, 60, 70, 80, 90, or 100% fewer false negative results than an otherwise similar method wherein the lateral flow assay strip does not comprise a trap region; and/or

the level of nucleic acid in a fluid sample after it has flowed through the trap region is decreased by at least 1, 5, 10, 20, 30, 40, 50, 60, 70, 80, or 90% relative to the level of nucleic acid present in the fluid sample prior to flowing through the trap region.

23. The lateral flow assay strip or method of paragraph 22, wherein the plurality of positively charged moieties bind to one, two, three, or all of the labeled target amplification products, labeled control amplification products, labeled unextended primers, or unlabeled primers.
24. The lateral flow assay strip or method of any of paragraphs 1-23, wherein the trap region is about 10-25 millimeters (mm) in length.
25. The lateral flow assay strip or method of any of paragraphs 1-24, wherein the trap region is about 3-7 millimeters (mm) in width.
26. The lateral flow assay strip or method of any of paragraphs 1-25, wherein the lateral flow assay strip or method has a detection range encompassing at least an approximately 50 fold range of analyte concentrations.

### EXAMPLES

### Example 1: A Rationale for Depletion of Labeled Material

Lateral flow immunoassays are simple to use diagnostic assays that can quickly and visibly report the presence or absence of a target analyte (e.g. a virus or pathogen). Thus, they are attractive and commonly used components of affordable point-of-care diagnostic tests. Nucleic acid lateral flow immunoassays (NALFIA) are used specifically to detect nucleic acid amplicons that carry one or more chemical labels. Prior to performing NALFIA, amplicons are commonly diluted in a lateral flow “running buffer.” Alternatively, a small volume of amplicons is loaded on the lateral flow strip and subsequently “chased” with a larger volume of lateral flow running buffer. Although pre-mixing or chasing sample with running buffer results in optimal lateral flow performance, this “dilution” step adds an extra step to the diagnostic workflow and requires one or more fluid transfer steps, often involving small volumes. Such dilution steps can be problematic for users in home or point-of-care settings, where precise and accurate volume transfers are cumbersome or not possible.

The hook effect, or high dose effect, is a well known immunological occurrence in which the formation of immune complexes is hindered by the high concentration of either the antibody or the antigen in the solution. This hook-like phenomenon can cause false-negatives, is most prevalent in one-step sandwich immunoassays, and is commonly overcome by dilution of the sample.

Without wishing to be bound by theory, it is thought that an excess concentration of shorter single-labeled primers from, e.g., LAMP (Loop-mediated isothermal amplification) reactions is thought to out-compete the longer double-labeled primers at the capture antibody sites, saturating the available binding sites on the lateral flow assay strip, and creating a hook effect. Additionally, a similar excess of unextended biotinylated primers may exhaust the supply of streptavidin-conjugated colloidal gold particles, limiting the ability to detect dual-labeled amplicons immobilized on the capture antibodies of an exemplary lateral flow assay strip. Low signal intensity on the lateral flow assay strips using undiluted LAMP reaction was observed, and signal intensity was increased by using a 5-14× dilution of the LAMP reaction. A modest increase in detection was obtained by doubling the amount of conjugate added to an undiluted LFA (Lateral Flow Assay) test. The following examples demonstrate solutions for mitigating or eliminating the hook effect from exemplary nucleic acid immunoassays for detecting SARS-CoV-2, which can adversely lead to a decrease in signal on the lateral flow test strip.

### Example 2: Solution Phase Depletion of Labeled Material

This example demonstrates the principle of solution-phase depletion of labeled material in a fluid sample prior to contacting a lateral flow assay strip with the fluid sample.

It was hypothesized that the hook effect is driven by an excess of hapten-labeled material (both unextended primers and primer-incorporated product). One potential solution is to reduce the overall concentration of all labeled material. This is the net effect of dilution: the concentration of both primers and products are reduced by a constant amount. Indiscriminate reduction of labeled material can be achieved in solution or via solid phase methods. For a solution-phase approach, an exemplary trap reagent, a solution-phase antibody, is added to the reaction mix before the sample is applied to the lateral flow assay (LFA) strip. The solution-phase antibody binds to the hapten labeled primers and product, rendering them incapable of binding to the immobilized antibodies on the LFA strip lines. A molar excess of solution-phase antibody relative to the hapten in solution may result in a complete suppression of any binding in the LFA strip line, but through careful titration of the antibody, an appropriate level of hapten reduction is achievable to mitigate the hook effect without completely suppressing binding of hapten to the LFA strip line, generating the same effect as dilution. The first exemplary trap reagent (the solution-phase antibody) may be accompanied by a second exemplary trap reagent, solution phase streptavidin, to similarly block biotin labeled products and primers from depleting the conjugated colloidal gold supply.

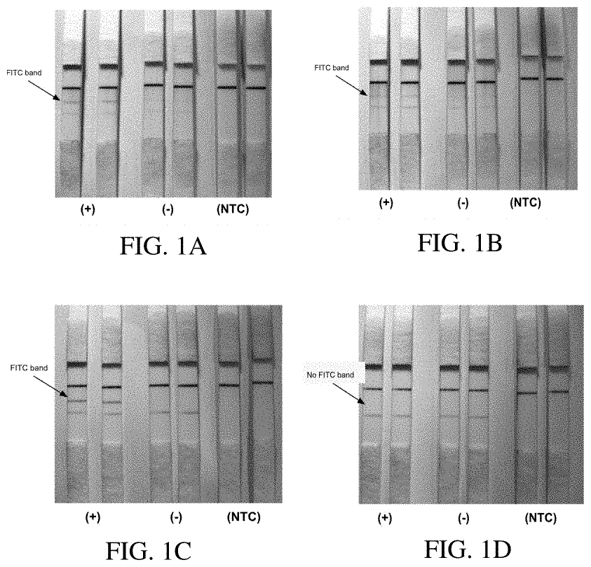

This concept was tested by contacting LAMP samples (diluted or undiluted) with a solution of antibodies to the exemplary hapten FITC and running the samples on exemplar LFA strips. Lateral flow assay strips were run in duplicate (left to right): LAMP reaction amplifying nucleic acid from a contrived COVID positive sample (+), LAMP reaction failing to amplify nucleic acid from a contrived healthy RNAseP negative sample (−), and LAMP reaction containing no template nucleic acid (No Template Control (NTC)), were applied to ABN lateral flow assay strips (FIGS. 1A-1D). The level of solution-phase anti-FITC mAb used did not reduce or eliminate FITC-labeled amplicon signal from undiluted LAMP samples (FIGS. 1A & 1B), but did remove all visible FITC-labeled amplicons in the diluted LAMP sample (FIGS. 1C & 1D). These data suggest that the amount of solution-phase antibody was insufficient to impact the amount of hapten-labeled DNA in an undiluted reaction, but it was sufficient to impact signal strength in diluted samples. This demonstrates that a solution phase trap reagent can bind to exemplary analytes and reduce/eliminate visible signal in an LFA; however, a suitable reduction Additional titration studies are performed to determine the appropriate concentration or range of concentrations of solution-phase antibody to mitigate the hook effect without completely suppressing binding of hapten to the LFA strip line. Also, these results further demonstrate the benefit of reducing the concentration of both the capture hapten (in this case, FITC) and visualization hapten (in this case, biotin). In this case, while the concentration of capture hapten (FITC) was reduced, the concentration of visualization hapten (biotin) was not reduced. This resulted in undiluted LAMP samples contacted with mAb to FITC, where the undiluted level of amplicons had freely available visualization hapten but a reduced level of amplicons had available capture hapten. Upon contact with an LFA strip, colloidal gold would label all amplicons with equal probability, resulting in “invisible” (capture hapten-obscured) amplicons uselessly binding much of the colloidal gold. As a result, the colloidal gold concentration was consumed at the undiluted concentration, and likely limited visualization.

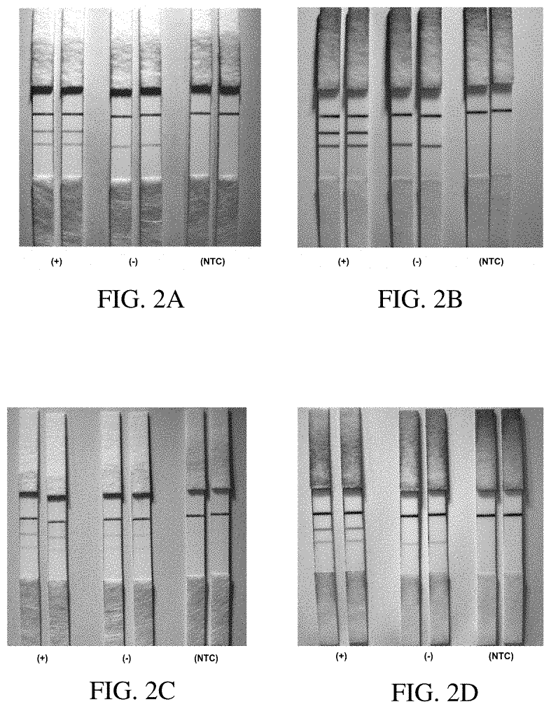

The impact of modulating the level of conjugated colloidal gold is demonstrated in FIGS. 2A-2D. Lateral flow assay strips were run in duplicate (left to right): LAMP reaction amplifying nucleic acid from a contrived COVID positive sample (+), LAMP reaction failing to amplify nucleic acid from a contrived healthy RNAseP negative sample (−), and LAMP reaction containing no template nucleic acid (No Template Control (NTC)), were applied to lateral flow assay stripsExperiments where an excess of 40 nm gold nanoparticles conjugated to streptavidin (at OD 10) was added to the LFA strip resulted in an increase in visible signal for both diluted LAMP (FIG. 2B) and undiluted LAMP (FIG. 2D) reactions relative to typical streptavidin-gold conjugate concentrations (FIGS. 2A & 2C). This demonstrates that, in the absence of excess streptavidin-gold conjugates, the undiluted and diluted LAMP samples contained sufficient visualization hapten (biotin) to consume available streptavidin-gold conjugates. It was observed that additional gold can create more background noise (e.g., by binding non-specifically to the strip) and may require additional buffer, e.g., 80 μL of buffer, to clear (data not shown).

These experiments indicated that with titration of trap reagents, e.g., solution phase antibodies, targeting one or both of the capture or the visualization haptens, solution phase partial depletion of labeled material could be used to increase the intensity of the signal in nucleic acid detection methods without a dilution step. They also demonstrated that addition of a second trap reagent, e.g., solution-phase streptavidin, could provide a reduction in biotin-labeled primers and products for similar effect.

### Example 3: Solid Phase Depletion of Labeled Material

This example demonstrates the principle of solid phase depletion of labeled material in a fluid sample as part of contacting a lateral flow assay strip with the fluid sample.

Although addition of solution phase antibody described in Example 2 may be effective, the mechanics of precisely and accurately combining a fluid sample with a trap solution may be problematic for users in point-of-care or at home settings. An alternative implementation of one or more trap reagents in the design of the lateral flow assay strip itself may have specific ease-of-use, reliability and robustness advantages.

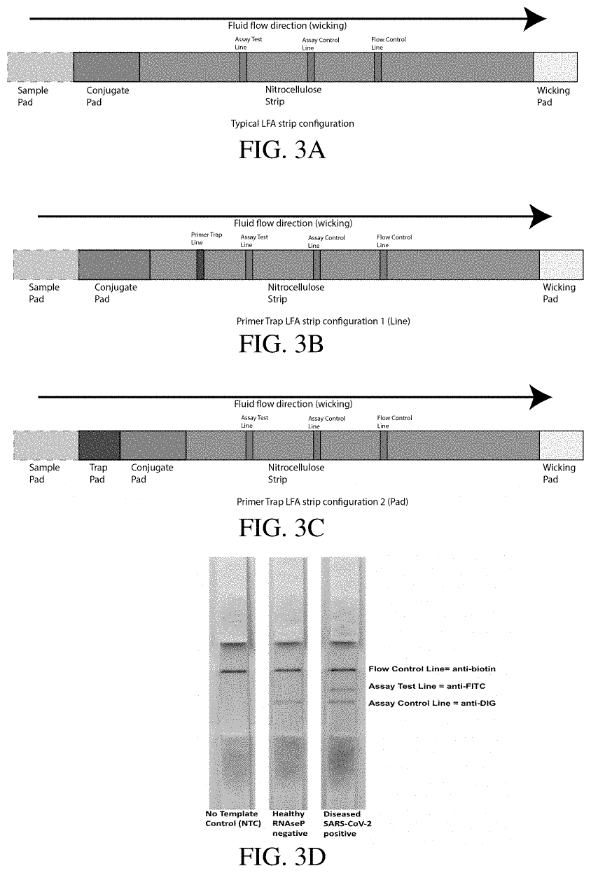

Rather than adding solution phase antibodies (e.g., via a trap solution), the one or more trap reagents (e.g., antibodies) can be immobilized and integrated in the physical design of an exemplary lateral flow assay strip, creating a specific region (a trap region) to retain the hapten-labeled primers as a “primer trap”. As illustrated in FIGS. 3A-3C, in an exemplary traditional lateral flow assay strip design (FIG. 3A), sample is either added to a specialized sample pad or directly to the conjugate pad. The sample pad (light blue dashed box) is optional; in lateral flow assay strip designs that lack a sample pad, samples can be added directly to the conjugate pad. When the sample encounters the conjugate pad, lyophilized streptavidin-conjugated colloidal gold is rehydrated by the wicking liquid front, and binds to the biotinylated products and unextended primers. As described above, an excess of biotinylated product in an undiluted sample can deplete the conjugate, leaving the majority of the biotinylated product without bound colloidal gold particles, and hence invisible.

As described in FIGS. 3B & 3C, a trap region may be employed, where exemplary trap reagents, the antibodies specific to the capture haptens for all reactions (control and test), are immobilized. In FIG. 3B, the trap region, either as a dispensed line on the existing lateral flow assay strip membrane or as a specific capture pad (a trap pad), partially depletes labeled product after the conjugate has bound to the biotinylated products. While this (in a printed Primer Trap Line format) may be easy to implement and integrate into existing manufacturing pipelines (e.g., in a printed trap line format that is printed in addition to test or control lines), it may not be as effective at increasing visualization of undiluted samples as the conjugate is still consumed by the excess product during the conjugate binding step. The use of a trap pad (or one or more dispensed trap lines) prior to the conjugate pad (see, e.g., FIG. 3C) may be advantageous, where the hapten-labeled primers and products in the undiluted sample are partially depleted before entering the conjugate pad, ensuring a higher ratio of conjugate to hapten-biotin labeled product. Such a solid-phase trap region can also comprise an additional trap reagent, e.g., immobilized streptavidin. For example, the immobilized streptavidin could also be included with one or more capture antibodies in a trap pad, thereby reducing the concentration of biotinylated and hapten-labeled primers and product prior to binding to the colloidal gold conjugate.

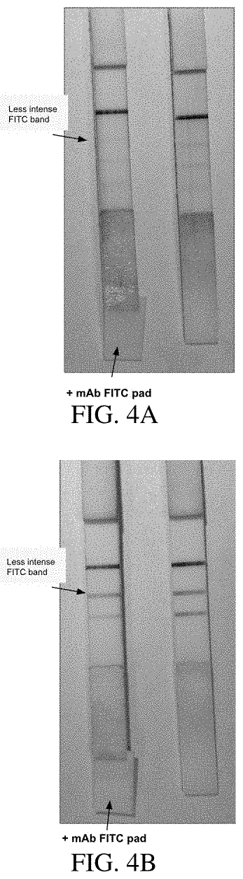

Experiments demonstrating the principle of the solid phase trap region are shown in FIGS. 4A & 4B. In this experiment, two strips were prepared: one where 2 μg anti-FITC mAb was dried onto a trap pad upstream of the conjugate pad—an exemplary implementation corresponding to the schema of FIG. 3C (left strips in FIGS. 4A & 4B); and one without the trap pad (right strips). Sample passage through the trap pad removed or reduced signal intensity in the FITC capture band in undiluted (FIG. 4A) and diluted (FIG. 4B) LAMP COVID positive samples, presumably due to reduced concentration of FITC-labeled primers and products. This data shows that a trap region, e.g., a trap pad, comprising one or more trap reagents can capture labeled target amplification product, labeled control amplification product, and/or labeled unextended primer prior to the products or primer encountering the one or more test lines of a lateral flow assay strip.

### Example 4: Using Buffer Components to Alter Fluid Sample pH

This example demonstrates that the pH of a fluid sample flowing through an exemplary lateral flow assay strip changes over time and indicates that pH modification may be an effective means to improve the detectability of test and control line signals.

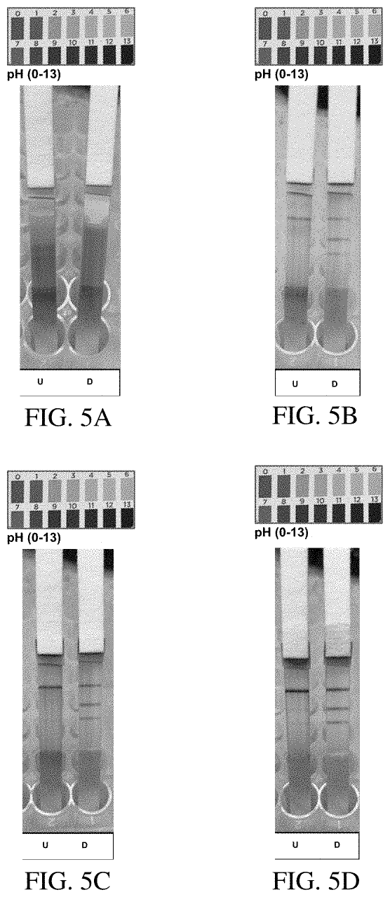

Samples are typically pre-diluted before addition to a lateral flow assay strip. Pre-dilution not only reduces the concentration of oligonucleotides, but can change the chemical composition of the sample as well. For samples comprising nucleic acid amplification reaction mixtures, the process of amplification introduces changes to the reaction mix, including potential shifts in pH as nucleotides are incorporated and H+ ions released. The potential impact of dilution buffer on the reaction samples is illustrated in FIGS. 5A-5D, where pH paper was placed under the wicking paper to indicate the sample pH for diluted COVID positive (+) LAMP in buffer (right) and undiluted COVID positive (+) sample in collection buffer (left), following sample application to the lateral flow assay strip over time (A)-(D).

| Lateral Flow Assay Strip | Observed pH |
|---|---|
| FIG. 5A (U) - Undiluted sample at 1 minute | 6 |
| FIG. 5A (D) - Diluted sample at 1 minute | 6 |
| FIG. 5B (U) - Undiluted sample at 3 minutes | 6 |
| FIG. 5B (D) - Diluted sample at 3 minutes | 5 |
| FIG. 5C (U) - Undiluted sample at 6 minutes | 6-7 |
| FIG. 5C (D) - Diluted sample at 6 minutes | 6 |
| FIG. 5D (U) - Undiluted sample at 9 minute | 7-8 |
| FIG. 5D (D) - Diluted sample at 9 minute | 7-8 |

These data indicate that the initial pH at 3 minutes (FIG. 5B) was higher for undiluted LAMP (left) relative to diluted LAMP (pH 4-5), but that after 9 minutes both samples had stabilized at a pH of 7-8 (FIGS. 5C & 5D).

These data indicate that the pH may be lower as the sample front initially moves through the conjugate and capture pads. The lower pH may provide suboptimal binding conditions for either the capture antibodies or the streptavidin conjugate. This could be addressed by providing one or more buffer components in solid form (e.g., lyophilized) in the sample pad and/or conjugate pad, adjusting the sample conditions to those optimal for antibody and streptavidin binding.

### Example 5: Using Extension Blocking Groups to Deplete Labeled Material

This example describes using trap reagents against extension blocking groups to partially deplete a fluid sample of labeled primers or amplification products to improve the signal of a subsequent immunoassay.

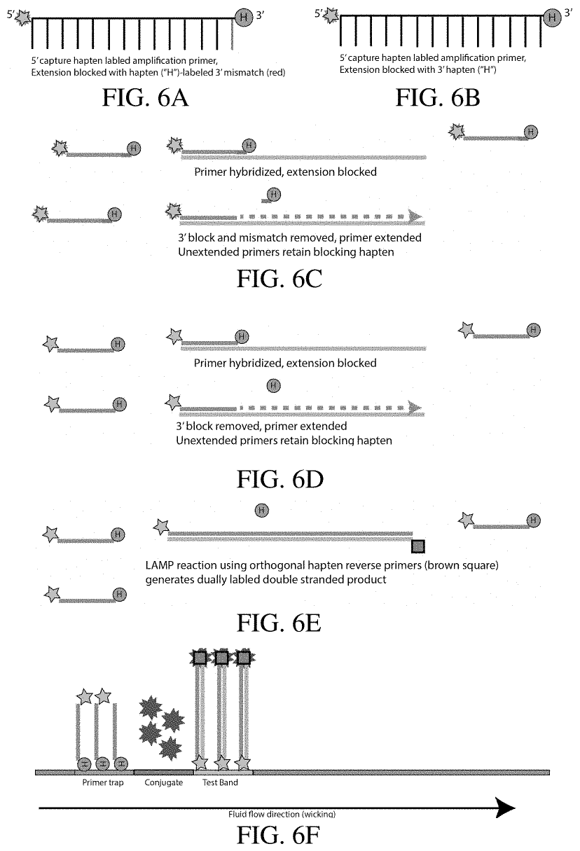

The use of extension blocking groups on the 3′ end of oligonucleotide primers has been described previously as an attempt to reduce non-specific amplification. (Ullman, E. F., et al. (2002). Method for polynucleotide amplification, U.S. Pat. No. 6,482,590 B1; Ankenbauer, W., et al. (2006). Composition and method for Hot Start nucleic acid amplification, US 2003/0119150 A1). As shown in FIGS. 6A-6F, this approach can be adopted as a means to selectively deplete unextended primers from a sample (star: 5′ capture hapten; blue encircled H: 3′ hapten (which may block extension); red base: mismatched base or unextendible nucleotide/abasic site; brown squares and starbursts: conjugate label). In one embodiment, a specific hapten is placed on a primer's extension blocking group (e.g., a 3′ blocking base (e.g., a mismatched base, non-complementary to the template, or an unextendable nucleotide analogue or abasic site, as in FIG. 6A)), or the hapten itself is utilized as the extension blocking group (e.g., as in FIG. 6B). Polymerases with exonuclease activity, or exonucleases used in addition to a polymerase, remove the extension blocking group, whether hapten or hapten+blocking base, for primer extension and nucleic amplification to occur. Unextended primers in the amplification reaction would retain the extension blocking group, whereas amplification products generated by exonuclease activity would lack the extension blocking group (FIGS. 6C & 6D).

This strategy would enable the use of a trap region on the lateral flow assay strip comprising one or more trap reagents that specifically target the extension blocking group. For example, using antibodies specific to the hapten of an extension blocking group (whether free-floating and liberated from the primer, or still attached to the unextended primer), the trap region of the lateral flow assay strip would pull labeled unextended primers retaining the extension blocking group from solution as shown in the blue region of the lateral flow assay strip in FIG. 6F. By removing the labeled unextended primers prior to exposing the amplicons to the conjugate pad (the brown region of the exemplary lateral flow assay strip in FIG. 6F), the ratio of conjugate to conjugate-specific hapten (the brown square) is increased, improving visibility of the conjugate labeled haptens (green extended products attached to brown starbursts in FIG. 6F). In addition, by removing the labeled unextended primers prior to exposing the amplicons to the conjugate pad, primarily labeled target amplification products and/or labeled control amplification products reach the conjugate pad and are labeled for detection. The same extension blocking group can be utilized for one or more (e.g., all) labeled primers for a given amplicon (e.g., the primer with the 5′ capture hapten as well as the primer with the 5′ detection hapten), such that a trap region comprising a trap reagent that binds a single extension blocking group can decrease the level of labeled unextended primer for multiple (e.g., all of the) different primers in a given amplification reaction. Similarly, the same extension blocking group can be used across multiple primer sets used in multiplex reactions (e.g., the primers for COVID-19 and those for human RNAseP controls), with the same advantage of targeting many or all primers with a single trap region or trap solution. In some embodiments, the same extension blocking group (e.g., 3′ blocking hapten) is used for all primers and a trap region comprising one or more trap reagents binds to said extension blocking group. In some embodiments, different primer- or product-specific extension blocking groups are employed and different trap reagents (e.g., disposed in one or multiple trap regions) specific to each of those extension blocking groups could be employed. Additionally, while the exemplary trap reagents are shown here as antibodies directly applied to the lateral flow assay strip, any manner of solid phase trap reagent could be utilized. For example, a trap reagent comprising an antibody could be applied to beads, other microparticles, or the walls of the sample chamber.

### Example 6: Using Trap Reagents Comprising Oligonucleotide to Deplete Labeled Material

This example describes using trap reagents comprising oligonucleotides with complementarity to one or more of labeled unextended primers, labeled target amplification products, or labeled control amplification products to partially deplete a fluid sample of labeled primers and/or product.

Depletion of hapten-labeled primers and product in undiluted samples could be accomplished with a trap region (e.g., trap pads or lines) comprising immobilized oligonucleotides complementary to one or more amplification primers. Melting temperatures for the capture primers would reflect the room temperature conditions. The trap reagent oligonucleotide(s) would bind to unincorporated primers as well as any single stranded product. As the latter lack a detection hapten-labeled complementary strand, this can advantageously reduce the amount of nondetectable amplicons competing for binding space in the test or control lines of exemplary lateral flow assay strips. In some embodiments, a trap reagent comprising an oligonucleotide is situated upon a high surface area substrate, e.g., dendrimers or crenulated microparticles.

### Example 7: Using Solution-Phase Size Exclusion or Ion-Exchange Resin, or Solid-Phase Charged Membranes, to Deplete Labeled Material

This example describes contacting a sample with a trap solution comprising exemplary trap reagents (e.g., size-exclusion or ion exchange resin) prior to applying the treated sample to a lateral flow assay strip to partially deplete the sample of one or more of labeled unextended primers, labeled target amplification products, or labeled control amplification products to partially deplete a fluid sample of labeled primers and/or product.

A further method to decrease the concentration of labeled unextended primers in the undiluted sample prior to contacting the lateral flow assay strip with sample utilizes size exclusion chromatography resin or ion exchange resin. The undiluted sample mix would run through a bed of resin prior to reaching the lateral flow assay strip. As the labeled unextended primers are smaller than extended labeled target amplification products and labeled control amplification products, porous media with appropriately sized pores will retard the movement of the labeled unextended primers, as they enter the beads and become trapped. The larger extended products are too large to enter the pores and travel around the beads, migrating unimpeded to the lateral flow assay strip.

A further method to decrease the concentration of labeled unextended primers in the undiluted sample prior to contacting the lateral flow assay strip with the sample utilizes trap region comprising a charged membrane (e.g., a trap pad comprising a charged membrane).

Some examples of possible resins include G-50, Sephacryl S-200 and S-300 resins. Possible membranes include (DEAE) ion-change such as those sold by Bio-Rad, for example at: https://www.bio-rad.com/en-us/product/macro-prep-deae-resin?ID=b67f362d-de39-4eee-9b66-775834614dd2.

### Example 8: Using Enzymatic Digestion to Deplete Labeled Material

This example describes using one or more trap reagents comprising an enzyme to deplete an undiluted sample of single stranded labeled unextend primers, leaving double-stranded labeled target amplification products and labeled control amplification products whole and detectable.

Single stranded primers and products may be removed from undiluted samples via one or more enzymatic processes. In some embodiments, shrimp alkaline phosphatase, a single-stranded exonuclease (e.g., Exo I), or both shrimp alkaline phosphatase and a single-stranded exonuclease may be used (e.g. Shrimp Alkaline Phosphatase or Exo-SAP, available at https://www.thermofisher.com/us/en/home/life-science/sequencing/sanger-sequencing/sangersequencing-kits-reagents/exosap-it-per-product-cleanup.html). These enzymes may be used to digest signal stranded primers post-amplification, but preserve double-stranded DNA. Unextended primers or singly labeled LAMP products would be digested and only double stranded, dually labeled products would remain. In some embodiments, a trap solution comprises a trap reagent comprising an enzyme, and an undiluted sample is contacted with said trap solution prior to contacting a lateral flow assay strip with the treated sample. In other embodiments, a trap reagent comprising an enzyme is immobilized in a trap region of a lateral flow assay strip.

### Example 9: Using Cationic Cellulose to Deplete Excess Nucleic Acid

This example describes using a trap region comprising a plurality of positively-charged moieties to partially deplete a fluid sample of one or more of labeled unextended primers, labeled target amplification products, or labeled control amplification products to partially deplete a fluid sample of labeled primers and/or product.

Lateral flow assay strips were prepared comprising a first sub-region wherein the fluid sample can be introduced to the lateral flow assay strip. An exemplary absorptive substrate, cellulose, was used. The cellulose was contacted with an exemplary chemical reagent comprising a positively-charged moiety, glycidyltrimethylammonium chloride (GTMAC), in the presence of an exemplary base, NaOH. The result was covalently modifying the hydroxyl groups of the cellulose of the first sub-region with positively charged quaternary amines, thereby forming a trap region capable of binding nucleic acid.

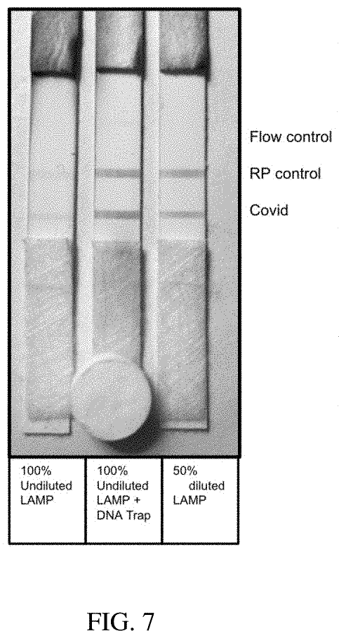

The target nucleic acid detection capabilities of lateral flow assay strips comprising the first sub-region comprising a trap region comprising a plurality of positively-charged moieties were compared to those of lateral flow assay strips not comprising a trap region when run with undiluted or diluted loop-mediated isothermal amplification (LAMP) samples (FIG. 7). In FIG. 7, the left lateral flow assay strip does not comprise an exemplary trap region comprising a plurality of positively-charged moieties in an exemplary first sub-region and was run with 100% undiluted LAMP sample; the middle lateral flow assay strip comprises an exemplary trap region comprising a plurality of positively-charged moieties in an exemplary first sub-region and was run with 100% undiluted LAMP sample; and the right lateral flow assay strip does not comprise an exemplary trap region comprising a plurality of positively-charged moieties in an exemplary first sub-region and was run with a LAMP sample diluted to 50%. When undiluted LAMP sample was run on a lateral flow assay strip that did not comprise an exemplary trap region, the signal intensity was faint. Signal intensity was much higher when undiluted LAMP sample was run on a lateral flow assay strip comprising the exemplary trap region, comparable to or exceeding the signal obtained when running a 50% diluted LAMP sample on a lateral flow assay strip not comprising the exemplary trap region.

This data demonstrates that a lateral flow assay strip comprising a first sub-region comprising a trap region comprising a plurality of positively-charged moieties can be used to obtain higher signal intensity, effectively solving the hook effect or high dose effect. The increase in signal intensity can improve detection of a target nucleic acid and subsequent diagnosis of a disease or condition in a subject. By obviating the need for a dilution step, a lateral flow assay strip (or device comprising the same) comprising a trap region of the disclosure has advantages in home care or point-of-care settings where fluid manipulation may be cumbersome or impossible.

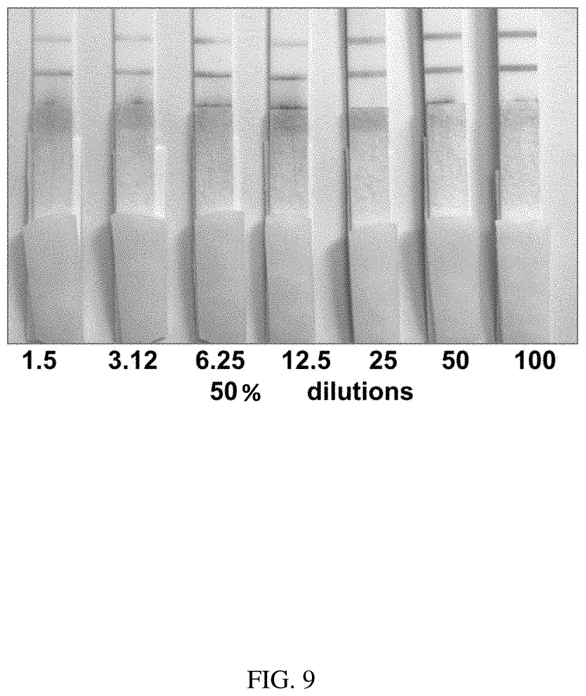

The detection range of lateral flow assay strips comprising the first sub-region comprising a trap region comprising a plurality of positively-charged moieties was evaluated. A LAMP sample was diluted by 50% volume in a series dilution. The diluted samples were applied to the first subregion of exemplary lateral flow assay strips, each comprising an exemplary trap region comprising a plurality of positively-charged moieties (cellulose comprising a plurality of ammonium groups, having been treated with GTMAC). The exemplary trap regions were 5 mm wide and 15-20 mm in length. When the samples were run on the lateral flow assay strips, visible signal was detectable over a wide range of concentrations, from 100% at right-most (undiluted) to 1.5% at left-most (an about 66 fold dilution), which represents an improvement over lateral flow assay strips not comprising trap regions (FIG. 9).

It was observed that increasing the length of the trap region (e.g., to 20 mm) increased the binding capacity of the trap region for nucleic acid and increased the detection range of the lateral flow assay strip (and a method for detecting a target nucleic acid using the lateral flow assay strip), relative to the 12 mm trap region length used in FIG. 7. In such a way, the trap region and a given lateral flow assay strip may be tuned to have a dynamic detection range encompassing an applicable analyte concentration level (e.g., where a longer trap region might enable visible signal detection for a sample containing a higher concentration of nucleic acid/analyte).

These results demonstrate that lateral flow assay strips containing trap regions comprising a plurality of positively-charged moieties produce visible signal detection over a nearly 70 fold concentration range of analyte, and that the detection range is tunable by, e.g., adjusting the length of the trap region.

### Example 10: Comparing Different Cationic Celluloses to Deplete Excess Nucleic Acid

This example describes using two different trap regions comprising two different pluralities of positively-charged moieties to partially deplete a fluid sample of one or more of labeled unextended primers, labeled target amplification products, or labeled control amplification products.

Lateral flow assay strips were prepared comprising a first sub-region wherein the fluid sample can be introduced to the lateral flow assay strip. An exemplary absorptive substrate, cellulose, was used. The cellulose was contacted with an exemplary chemical reagent, 2-chlorotriethylamine, in the presence of an exemplary base, NaOH. The result was covalently modifying the hydroxyl groups of the cellulose of the first sub-region with positively charged DEAE moieties, thereby forming a trap region capable of binding nucleic acid.

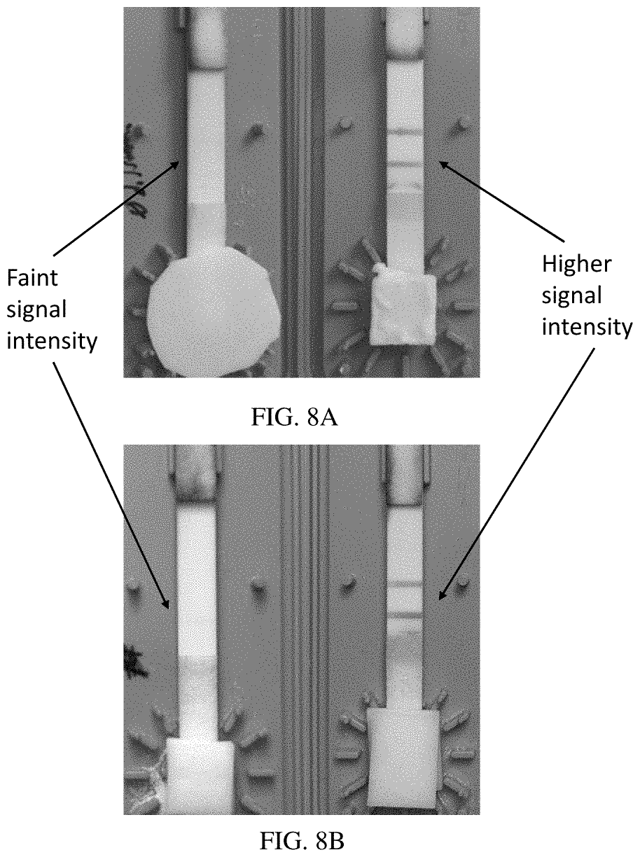

The target nucleic acid detection capabilities of lateral flow assay strips comprising the first sub-region comprising a trap region comprising a plurality of positively-charged moieties were compared to those of lateral flow assay strips not comprising a trap region, as well as the exemplary lateral flow assay strips of Example 9, when run with undiluted or diluted loop-mediated isothermal amplification (LAMP) samples (FIGS. 8A and 8B). FIG. 8A shows at left a lateral flow assay strip not comprising a trap region, and at right a lateral flow assay strip comprising an exemplary trap region comprising a plurality of diethylaminoethyl (DEAE) moieties. FIG. 8B shows at left a lateral flow assay strip not comprising a trap region, and at right a lateral flow assay strip comprising an exemplary trap region treated with glycidyltrimethylammonium chloride (GTMAC) and base (e.g., NaOH 10%) and comprising a plurality of quaternary or tertiary amine groups. When undiluted LAMP sample was run on the lateral flow assay strips of FIGS. 8A and 8B that did not comprise an exemplary trap region, the signal intensity was faint. Signal intensity was much higher when undiluted LAMP sample was run on a lateral flow assay strip comprising the exemplary trap region comprising DEAE moieties. The exemplary lateral flow assay strip of Example 9 (treated with GTMAC) yielded comparable intensity improvements to the exemplary lateral flow assay strip comprising DEAE moieties.

This data further demonstrates that a lateral flow assay strip comprising a first sub-region comprising a trap region comprising a plurality of positively-charged moieties can be used to obtain higher signal intensity, effectively solving the hook effect or high dose effect. This data shows that a number of different positively charged moieties, including DEAE moieties, can be used to obtain higher signal intensity.

What is claimed is:

1. A lateral flow assay strip usable to identify at least one target nucleic acid, the lateral flow assay strip comprising:
an absorbent substrate having a first end and a second end, wherein the substrate is configured to move a fluid sample from the first end to the second end by capillary action, and
a plurality of sub-regions comprising:
a first sub-region wherein the fluid sample can be introduced to the lateral flow assay strip, wherein the first sub-region comprises a trap region comprising a plurality of positively charged moieties,
optionally a second sub-region comprising a particle conjugate pad, and
a third sub-region comprising one or more test lines, and
wherein the one or more test lines are configured to indicate the presence of at least one target nucleic acid by interaction with the fluid sample.

2. A method for detecting a target nucleic acid in a fluid sample, the method comprising:
(i) contacting a lateral flow assay strip with a fluid sample, wherein the lateral flow assay strip comprises:
an absorbent substrate having a first end and a second end, wherein the substrate is configured to move a fluid sample from the first end to the second end by capillary action, and
a plurality of sub-regions comprising:
a first sub-region wherein the fluid sample can be introduced to the lateral flow assay strip, wherein the first sub-region comprises a trap region comprising a plurality of positively charged moieties capable of binding nucleic acid,
optionally a second sub-region comprising a particle conjugate pad, and
a third sub-region comprising one or more test lines, and
wherein the contacting occurs under conditions under which a target nucleic acid or labeled target amplification product which may be present in the fluid sample can bind to the one or more test lines to produce a first detectable signal, and
(ii) identifying the presence of the target nucleic acid in the fluid sample based upon detecting the presence of the first detectable signal, or identifying the absence of the target nucleic acid in the fluid sample based on detecting the absence of the first detectable signal.

3. The lateral flow assay strip of claim 1, wherein the plurality of positively charged moieties comprise amino groups.

4. The lateral flow assay strip of claim 1, wherein the plurality of positively charged moieties comprise one, two, three, four, or all of:
an amino group comprising a trimethylamino, dimethylamino, or diethylaminoethyl (DEAE) group,
a heterocyclic group comprising a piperidinyl or morpholino group,
an aromatic group comprising an imidazolyl or pyridinyl group,
an imine group, or
a metal ion with a chelating group, wherein the metal ions comprise:
divalent cations selected from the group consisting of Ni²⁺, Zn²⁺, Cu²⁺, Ca²⁺, Co²⁺, and Fe²⁺;
trivalent cations selected from the group consisting of Al³⁺, Ga³⁺, Fe³⁺, and Cr³⁺; or
a lanthanide ion.

5. A method of preparing a lateral flow assay strip comprising a trap region,
wherein the lateral flow assay strip comprises:
an absorbent substrate having a first end and a second end, wherein the substrate is configured to move a fluid sample from the first end to the second end by capillary action, and a plurality of sub-regions comprising a first sub-region wherein the fluid sample can be introduced to the lateral flow assay strip;
the method comprising:
contacting the first sub-region with a chemical reagent comprising a positively charged moiety and/or exposing the first-sub region to suitable conditions for providing a positively charged moiety to the absorbent substrate, thereby providing a trap region in the first sub-region,
thereby preparing a lateral flow assay strip comprising a trap region.

6. The lateral flow assay strip of claim 1, wherein the absorbent substrate comprises a first polymer comprising a polysaccharide, a methyl acrylic polymer, a poly-lysine, or a styrenic polymer.

7. The lateral flow assay strip of claim 6, wherein the polysaccharide is selected from the group consisting of cellulose, dextrin, chitosan, chitin, agarose, and nitrocellulose.

8. The lateral flow assay strip of claim 6, wherein the polysaccharide comprises a plurality of sugar monomers, and wherein a plurality of the sugar monomers are chemically modified to comprise the plurality of positively charged moieties.

9. The lateral flow assay strip of claim 1, wherein the positively charged moieties were attached by contacting the first sub-region with a chemical reagent and/or exposing the absorbent substrate of the first sub-region to suitable conditions.

10. The lateral flow assay strip of claim 9, wherein the suitable conditions comprise alkaline or acidic conditions.

11. The lateral flow assay strip of claim 9, wherein the chemical reagent is selected from the group consisting of glycidyltrimethylammonium chloride (GTMAC) 2-chlorotriethylamine, and lysine.

12. The method of claim 5, wherein the contacting step further comprises contacting the first sub-region with a chemical cross-linker selected from the group consisting of a dihalohydrin and 1,4-butanediol diglycidyl ether.

13. The lateral flow assay strip of claim 6, wherein the first sub-region and/or trap region comprises a second polymer comprising polyethylenimine (PEI), poly-lysine, chitin, and/or chitosan.

14. The lateral flow assay strip of claim 1, wherein the first sub-region is thicker than at least one adjacent sub-region.

15. The lateral flow assay strip of claim 1, wherein the first sub-region and/or trap region comprises a trap pad comprising an absorbent substrate that is separate from the absorbent substrate of at least one adjacent sub-region while remaining in fluidic communication with at least one adjacent sub-region.

16. The method of claim 2, further comprising:
performing an isothermal amplification reaction on the biological sample, wherein the biological sample comprises a target nucleic acid,
thereby producing an amplification product mixture comprising one, two, three, or all of the labeled target amplification products, labeled control amplification products, labeled unextended primers, or unlabeled primers.

17. The method of claim 2, wherein:
i. the first detectable signal, second detectable signal, or both are at least 10, 20, 30, 40, 50, 60, 70, 80, 90, 100, 200, or 300% more intense than the signal that would be obtained in an otherwise similar method wherein the lateral flow assay strip does not comprise a trap region;
ii. the method produces at least 10, 20, 30, 40, 50, 60, 70, 80, 90, or 100% fewer false negative results than an otherwise similar method wherein the lateral flow assay strip does not comprise a trap region;
iii. the level of nucleic acid in a fluid sample after it has flowed through the trap region is decreased by at least 1, 5, 10, 20, 30, 40, 50, 60, 70, 80, or 90% relative to the level of nucleic acid present in the fluid sample prior to flowing through the trap region; or
iv. a combination of one, two, or all of i.-iii.

18. The method of claim 16, wherein the plurality of positively charged moieties bind to one, two, three, or all of the labeled target amplification products, labeled control amplification products, labeled unextended primers, or unlabeled primers.

19. The lateral flow assay strip of claim 1, wherein the trap region is about 10-25 millimeters (mm) in length and/or about 3-7 millimeters (mm) in width.

20. The method of claim 2, wherein the lateral flow assay strip has a detection range encompassing at least an approximately 50 fold range of target nucleic acid concentrations.
